(19) United States
(12) Patent Application Publication
(10) Pub. No.: US 2026/0317718 A1
(43) Pub. Date: Oct. 15, 2026

(54) SYSTEMS AND METHODS FOR COMMERCIAL SCALE CULTURED ADIPOSE TISSUE PRODUCTION

(71) Applicant: Trustees of Tufts College, Medford, MA (US)

(72) Inventors: David Kaplan, Medford, MA (US); John Se Kit Yuen, Medford, MA (US)

(21) Appl. No.: 19/101,395

(22) PCT Filed: Aug. 4, 2023
(86) PCT No.: PCT/US2023/071704

§ 371 Date: Feb. 5, 2025

Related U.S. Application Data
(60) Provisional application No. 63/370,641, filed on Aug. 5, 2022.

(51) Int. Cl. C12N 5/077; C12M 1/00

(52) U.S. Cl. CPC C12N 5/0653; C12M 47/02; C12N 2500/14; C12N 2500/30; C12N 2500/34; C12N 2506/1384; C12N 2513/00

(57) ABSTRACT

The present disclosure relates to cultured adipose tissue. In one embodiment, the cultured adipose tissue is produced by culturing adipose cells in a culture media in vitro, harvesting non-adhered adipose cells after a desired amount of non-adhered adipose cells are produced, and aggregating the harvested non-adhered adipose cells to provide the cultured adipose tissue. In some embodiments, aggregating the harvested non-adhered adipose cells comprises mixing the harvested non-adhered adipose cells with a hydrogel or binder in a three-dimensional (3D) mold. In other embodiments, aggregating the harvested non-adhered adipose cells comprises cross-linking the harvested non-adhered adipose cells in a 3D mold. In some embodiments, aggregating the harvested non-adhered adipose cells comprises flocculation or coagulation of the harvested non-adhered adipose cells. The cultured adipose tissue may have a defined 3D shape and a size on the macroscale. In some embodiments, the cultured adipose tissue may be a food product.

### CROSS-REFERENCE TO RELATED APPLICATIONS

This application claims priority to U.S. Provisional Application No. 63/370,641 filed on Aug. 5, 2022, the contents of which are incorporated by reference in their entirety.

### STATEMENT REGARDING FEDERALLY FUNDED RESEARCH

This invention was at least partially made with government support under 1954-1558 awarded by Advanced Research Projects Agency-Energy by the U.S. Department of Energy. The government has certain rights in the invention.

### FIELD OF INVENTION

The present disclosure generally relates to cultured adipose tissue produced on a macroscale level. The present disclosure further relates to methods for producing cultured adipose tissue on a macroscale level via the harvesting and aggregation of non-adhered adipose cells. The present disclosure also considers the use of flocculation and coagulation techniques in the aggregation of adipose cells.

### BACKGROUND

Conventional animal agriculture for the production of meat (muscle and fat tissue) is linked to numerous drawbacks such as environmental degradation, zoonic disease emergence, antimicrobial resistance, and animal welfare concerns. To provide the world with alternatives to animal products having reduced negative impacts on animals and the environment, there is increasing interest in producing cultured (in vitro) fat tissue.

In the field of alternative proteins, existing solutions to recapitulate the fat content of meat largely revolve around the direct addition or utilization of plant fats and oils (e.g., coconut oil). Recent developments in this realm include the use of oleogels, where nanoscale globules of vegetable oils are generated to better create the texture of solid fats (i.e., animal fats). However, plant-based fats do not incorporate the often complex aroma and flavor of meat, as well as species-specific flavors that distinguish meat from cows, pigs, chicken, fish, and so on.

Native (in vivo) adipose tissue is largely a dense packing (aggregation) of lipid-filled adipocytes held together by a sparse extracellular matrix (ECM). This is opposed to muscle tissue which is comprised of aligned fibers in a multi-hierarchical structure. To date, three-dimensional (3D) culture has been the main approach for generating bulk/macroscale tissues. These tissue engineering strategies involve the in vitro growth of cells over 3D scaffolds. However, it is challenging to scale up 3D culture due to mass transport limitations with regard to oxygen, nutrients, and waste. It is often quoted in the field that cells cannot remain viable in 3D tissues unless they are within about 200 micrometers of a source of blood or culture media. Overcoming these challenges to maintain cell viability in 3D tissues may require vascularization or the incorporation of an elaborate tissue perfusion system to distribute nutrients to the cells. It is currently infeasible to directly grow large tissues on the macroscale (millimeter scale and up) using contemporary tissue engineering techniques without the use of perfusion or related methods or with the structural features of fat as found in vivo. Likely due to these challenges, large-scale production of adipose tissue that mimics those found in vivo does not appear to have been implemented to date.

Thus, there remains a need for strategies for the large-scale production of cultured adipose tissue. The present disclosure provides a technical solution to this need.

### SUMMARY

In certain embodiments, methods are provided in this disclosure. In one embodiment of broad aspects, a method for producing cultured adipose tissue is provided. The method may include the steps of: growing adipogenic precursor cells in a first culture media; differentiating the adipogenic precursor cells to adipose cells in a second culture media; harvesting non-adhered adipose cells; and aggregating the harvested non-adhered adipose cells to provide a cultured adipose tissue. The first culture media and the second culture media may optionally be the same media.

In another embodiment, a method for producing cultured adipose tissue is provided. The method may include the steps of: growing adipogenic precursor cells on a two-dimensional (2D) substrate; differentiating the adipogenic precursor cells to adipose cells on the 2D substrate; harvesting non-adhered adipose cells; and aggregating the harvested non-adhered adipose cells to provide the cultured adipose tissue.

In certain embodiments, cultured tissues are provided. In one such embodiment, a cultured adipose tissue has adipose cells embedded in a hydrogel or binder, wherein the cultured adipose tissue has a three-dimensional (3D) shape and a size on the macroscale. In another embodiment, a cultured adipose tissue is made by the methods described above.

Additional objects, aspects, features, and advantages of this disclosure are provided in greater detail below.

### BRIEF DESCRIPTION OF THE DRAWINGS

The foregoing and other objects, aspects, features, and advantages of the present disclosure will become more apparent and better understood by referring to the following description taken in conjunction with the accompanying figures in which:

FIG. 1 is a schematic representation of cultured adipose tissue, in accordance with the present disclosure.

FIG. 2 is a flow chart of steps that may be involved in producing the cultured adipose tissue, in accordance with the present disclosure.

FIG. 3 is a schematic representation of methods of producing the cultured adipose tissue using bioreactors, in accordance with the present disclosure.

FIG. 4 is a schematic representation of a continuous process of producing the cultured adipose tissue on a conveyor belt, in accordance with the present disclosure.

FIG. 5 is a timeline of 3T3-L1 adipogenic differentiation, according to an embodiment of the present disclosure.

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When ranges for a particular value are recited, this disclosure contemplates all combinations of each of the upper and lower bounds of those ranges, including each of the values within those ranges, both individually and in a range. For example, recitation of a value of from 1 to 10 also contemplates a value of from 1 and 9 or from 3 and 10. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “adipogenic precursor cells” or “pre-adipocytes” refer to precursor cells capable of differentiating into mature adipose cells. “Adipogenic precursor cells” or “pre-adipocytes” may be used interchangeably throughout the present disclosure. Non-limiting examples of adipogenic precursor cells include stem cells such as pluripotent stem cells (PSCs), mesenchymal stem cells (MSCs), muscle-derived stem cells (MDSCs), and adipose-derived stem cells (ADSCs) (e.g., porcine, bovine, human, avian (chicken), piscine, etc.). In addition, transdifferentiated cells can also be utilized. Other adipogenic precursor cells may include, but are not limited to, dedifferentiated fat (DFAT) cells (e.g., porcine, bovine, piscine, etc.), preadipocytes (e.g., human, bovine, avian (chicken), murine, piscine, etc.), and fibroblasts (e.g., avian (chicken), bovine, porcine, murine, piscine, etc.).

As used herein, “adipose cells” are fat cells or adipocytes. “Adipose cells”, “fat cells”, and “adipocytes” may be used interchangeably throughout the present disclosure.

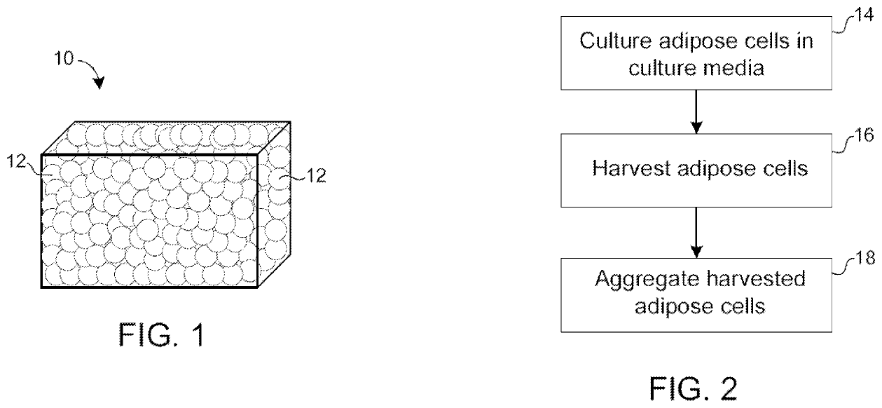

Referring now to the drawings, and with specific reference to FIG. 1, a schematic representation of cultured adipose tissue 10 is shown. The cultured adipose tissue 10 may include adipose cells 12 (or adipocytes 12) in an extracellular matrix. The cultured adipose tissue 10 may be arranged in a defined three-dimensional (3D) shape and may have a size on the macroscale (i.e., millimeter scale and greater). Although a cube-like structure is shown in FIG. 1 for simplicity, it will be understood that the cultured adipose tissue 10 may have any suitable 3D shape in practice. In some embodiments, the cultured adipose tissue 10 may be a food product suitable for consumption. In other embodiments, the cultured adipose tissue 10 may be incorporated as an ingredient in a food product suitable for consumption. As explained further below, the cultured adipose tissue 10 is produced using a method that circumvents the mass transport limitations associated with directly culturing bulk or large scale 3D tissues.

Turning to FIG. 2, a general exemplary method for producing the cultured adipose tissue 10 is shown. At a first block 14, a mass of adipose cells 12 (individual adipose cells 12, or small clusters of adipose cells 12) are cultured from adipogenic precursor cells in culture media. For instance, the block 14 may include growing adipogenic precursor cells to confluency (or to a desired coverage/number of cells on a surface or in suspension) in a first culture media, and then differentiating the adipogenic precursor cells into adipose cells 12 in a second culture media. The first culture media may be an adipogenic induction media which supports proliferation of the adipogenic precursor cells, and the second culture media may be a lipid accumulation media to provide large numbers of lipid-filled adipose cells 12. In alternative embodiments, a single culture medium may be used for both proliferation/growth of the adipogenic precursor cells and for differentiation of the adipogenic precursor cells into adipose cells. The culture time may be tuned to control lipid yield and droplet size. For example, Applicant has found that longer culture times (about a month) yield droplets comparable to in vivo fat (e.g., chicken).

In some embodiments, the adipose cells 12 may be genetically modified. For example. the adipose cells 12 may be genetically modified to improve their growth and lipid accumulation for more efficient scale up. A skilled artisan will recognize the variety of techniques that allow suitable genetic manipulation, including but not limited to, gene editing technologies such as CRISPR-Cas9 gene editing.

When it comes to genetic modification, there are two important aspects of the present disclosure. First, the gene modification to generate a given desired property is clearly important. As a result, the methods and systems described herein may include gene editing facilities and methods. On the other hand, genetically modified cells are increasingly available, so it is likely that commercial suppliers for genetically modified cells will be available soon. Second, it may be important to confirm that a given genetic modification is present in a cell or population of cells. If the production process or facility operates based on an assumption that the cells will have a given genetic modification, it could be extremely wasteful to proceed with faulty starting materials.

With respect to the specific genetic modifications, there are a host of categories that are more conventional genetic modifications (e.g., nutritional content, flavor, etc.) and some that are less conventional (e.g., enhancing surface proteins to facilitate the crosslinking described herein).

In some cases, the genetic modification may be a genetic modification to induce adipogenesis. In these cases, the adipogenic gene modification can be selected from the group consisting of a modification for ectopic expression of: peroxisome proliferator-activated receptor gamma (PPARγ), CCAAT/enhancer binding protein alpha and beta (C/EBPα, β), sterol regulatory element binding protein 1 (SREBP-1), fatty acid binding protein 4 (FABP4), zinc finger protein 423 (Zfp423), perilipin 1 (PLIN1), Kruppel-like factor 13 (KLF13), retinoid X receptor α (RXRα), phosphoenolpyruvate carboxykinase 1 (PCK1), early B-cell factor (Ebf) 1-3, runt-related transcription factor 1 (RUNX1), cyclin-dependent kinase 4 (CDK4), combinations thereof, or the like.

In cases where improved growth is desired, genetic modifications such as those described in WO 2018/208628, which is incorporated herein in its entirety by reference, may be usable.

In some cases, it may be desirable to downregulate expression of certain cellular components. For example, given that the point of the system and methods here is making fat cells, we would like to prevent breakdown of fat, so the genetic modification can be a genetic modification to downregulate production of adipose triglyceride lipase (TGL), hormone sensitive lipase (HSL), carnitine palmitoyltransferase (CPT), other fat or fatty acid breakdown enzymes, combinations thereof, or the like.

In other cases, it may be desirable to genetically modify the cells to generate cellular components that are helpful in food preservation. For example, the genetic modification could cause the production of anti-microbial, anti-fungal, or anti-viral constituents. As another example, the genetic modification could cause the production of stabilizing molecules. In some cases, the stabilizing molecules can prevent protein aggregation (e.g., low molecular weight stabilizers).

In some cases, the genetic modifications are intended for health purposes. In one specific case, the genetic modification can cause expression of vitamins that are not typically present in the cells. In another case, the genetic modification can cause expression of resveratrol, the active ingredient in red wine, or catechins, the active ingredient in green tea.

In some cases, the genetic modifications are intended to alter the color of the resulting cultured adipose tissue. Consumer perceptions regarding the color of food can be important. To that end, the genetic modifications can include expression of proteins that provide desirable colors, such as carotenoids, to alter the color of the resulting product to be more appealing to the average consumer. In some cases, the genetic modification can prompt the generation of myoglobin or hemoglobin, which can provide a color that is more typically associated with meat.

Genetic modification to modify flavor can come in a wide variety, given the variety of flavors that are capable of being expressed. To give one specific example, it is believed that expressing higher levels of fatty acid desaturases (e.g., generators of omega 3 fatty acids) can also generate more arachidonic acid, which is strongly correlated with enhanced flavor.

The genetic modifications can be modifications to hasten proliferation (cell immortalization targets are generally applicable here), improve adipogenesis, improve cell growth (e.g., allow growth on a media that has less components or less expensive components), or other modifications that would be understood to improve the overall number and quality of adipose cells for incorporating into the cultured adipose tissue.

In some cases, genetic modification can be used to introduce components that are not produced within the fat cells of the species of interest. For example, cows are a good source of conjugated linoleic acids (CLAs), which are known to be good for human health, but these CLAs are derived from their gut bacteria metabolites, so in vitro cells may be lacking in them. The genetic modification may be a gene that is associated with the digestive bacteria of a species of interest.

In the realm of more conventional beef genetics, studies have been done correlating single nucleotide polymorphisms (SNPs) with desired phenotypes (e.g., flavor, fattiness, degree of marbling, etc.). These SNPs are particularly desirable targets for genetic modifications for the cells described herein. In some cases, SNPs identified in the literature may correlate to changes in specific proteins (i.e. may represent a genotype that correlates directly to the phenotype). The changes demonstrated to be favorable for conventional meat products can be incorporated into the cells of the present disclosure. Examples of relevant SNPs include, but are not limited to, a G instead of A at locus rs733003230 of the fatty acid desaturase 1 (FADS1) gene and/or a G instead of A at locus LC060926 of the FADS2 gene for increased arachidonic acid in chickens or a G instead of C at position 841 of the fatty acid synthetase gene for increased oleic acid in cattle.

In some cases, the genetic modifications can be intended to facilitate the systems and methods described herein. For example, in embodiments where cells are actually crosslinked to one another, the genetic modification can modify the density of surface proteins and/or surface binding sites to facilitate the crosslinking. In some cases, the surface chemistry can include photo-initiated crosslinking agents.

The end product of this system and method is a cultured adipose tissue that is typically going to be combined with some kind of muscle tissue or connective tissue, so it may be beneficial to genetically modify the cells to express muscle binding or connective tissue binding domains, which may make harvesting easier.

In order to confirm the presence of genetic modifications, it may be useful to analyze the cells prior to introducing them into the system and method described herein. Skilled artisans will appreciate how to validate the presence of these genetic modifications.

In some cases, the specific genetic modifications that are present may be unknown or may be unknowable. In other cases, the cells may have no specific genetic modification, but may simply be harvested from a new source with unknown genetic makeup. At some points, the systems and methods described herein may encounter cells with unknown genetic makeup.

When such cells are received having unknown genetic makeup, if measuring the genetic modification or genetic makeup is not possible or not fruitful (i.e., unknown genes or expression products), then the cells can be subjected to culture media sampling to determine the most efficient culture media for use with those cells. This process can be manual or it can be automated. In the manual process, a lab technician will attempt to grow cells in different culture media and will compare the results to determine a cost/benefit to the different culture media. In the automated process, an artificial intelligence or machine learning algorithm can sample the variable space with respect to culture media composition and can optimize the media to the specific cells.

It would be appreciated that this optimization process does not require unknown genetic makeup and can be applied to the operation of the system and method will full knowledge of the genetic makeup of the cells. Even with full genetic knowledge, it may be unclear which culture media is optimal, so the optimization process described above with respect to the unknown genetic makeup can be performed to optimize the performance with cells where the genetic information is known.

In certain cases, a pre-production process can be performed, where a subset of cells from a population are removed from the population and put through a small-scale version of the methods described herein, to determine suitability of the culture media. In some case, less than 1% of a population of cells, less than 0.5%, less than 0.1%, or less than 0.01% of a population of cells can be selected for sub-sampling to confirm culture media appropriateness prior to a fully scaled up production run. The pre-production process can serve as a final validation that the cells and the media are compatible with one another.

The first step/cellular raw materials may be referenced as a cell stock. The acquisition of the cell stock (i.e., cell isolation) can in general be a process similar to harvesting conventional meat products (i.e., butchery and isolation of cells from the butchered products) or can involve harvesting cells from less traditional places, such as other active cell lines that are being used in cellular agriculture. Additional processing steps for the cells can include immortalization, or clonal isolation to obtain a proliferative+adipogenic+genetically homogenous (for production consistency) cell population/cell line. Cells could also be adapted to single cell suspension prior to use in scaled up production.

The culture media used can be provided in a variety of forms, including powdered bases that can be hydrated by pure water and fully formed culture media. In either case, separate and distinct culture media can be included for the proliferation and differentiation steps.

The purity of the water is extremely important. In some cases, the system includes on-site water purification.

If the culture media is provided in powder form, the method can include hydrating the powder to form a culture media.

The culture media can be sterile filtered or sterilized via any means available to those of skill in the art prior to use in the method or at any step of the method.

Various contemplated modifications to the culture media include, but are not limited to, bulk mixed powder including all of the non-water components of the culture media, separately maintained ingredients that are mixed on demand to make culture media, growth factors or artificial mimics thereof can be present in the culture media, or a combination thereof.

In some cases, the cells are genetically modified to have intentionally less strict requirements for the culture media. For example, cells can be genetically modified to create their own glutamine (see, for example, WO 2019/014652, which is incorporated herein in its entirety by reference for all purposes), which would eliminate the requirement for glutamine in the culture media. Similar genetic modifications to reduce the need for complex culture media are contemplated.

Manipulation of the cells themselves can be done by conventional methods, including seed trains for seeding larger and larger bioreactors. The seed train gradually moves to bioreactors having increasing size until the desired bioreactor size is reached. From there, the cells can be a part of a suspension culture of an adherent culture.

For a suspension culture, without wishing to be bound by any particular theory, it is believed that single cell suspension cultures are the most viable way to scale up this suspension culture. In one embodiment, cell proliferation is done via cell stock cultured in single cell suspension in various suspension bioreactors. These bioreactors can be a conventional stirred suspension tank, air-lift, vertical wheel, or wave bioreactors. In some cases, cells can be encapsulated with a material that protects the cells from shear stress. This encapsulating material may itself provide a miniature three-dimensional cell culture environment, which can be advantageous for other reasons discussed elsewhere herein. Other contemplated bioreactor designs include the rotating wall vessel bioreactor described elsewhere herein.

Turning to non-suspension aspects, the bioreactor used herein can be a dedicated adherent cell bioreactor, such as a fixed bed bioreactor (e.g., the Scale X™ bioreactor, available commercially from Univercells S.A., Charleroi, Belgium, or iCellis® bioreactors, available commercial from Pall Corporation, Port Washington, NY). In some cases, the bioreactor can include stacked culture flask approaches, like “cell factories” or the Hyperstack®, available commercially from Corning Inc., Corning, NY.

In one specific aspect, an adherent approach could be combined with a suspension bioreactor if microcarriers (e.g., pectin microcarriers commercially available from Corning Inc., Corning, NY) can be used as the adherent substrate, but those microcarriers are then circulated in a suspension bioreactor in the conventional fashion. The microcarriers can be edible. The microcarriers may end up in the resulting product, if their presence is not undesirable.

Turning to the adipogenic differentiation portion of the process, the same reactor can be used as was used for proliferation, with a change of culture media facilitating the transition. In some cases, the cells are transferred to a different bioreactor to transition from proliferation to differentiation.

When using the same reactor, cells can be separated from the proliferation media via centrifugation (e.g., disc stack centrifuge, counter flow centrifuge, Gibco Rotea centrifuge, etc.). After removal of the proliferation media, the differentiation media is added to the bioreactor and the cells are cultured in the same fashion as they undergo adipogenesis.

One important consideration in this work is that adipocyte buoyancy in suspension culture poses unique problems that are not typically encountered in cell agriculture. In conventional cell agriculture, when a cell floats, that is typically a sign that the cell is no longer of any value. However, in this disclosure, the buoyancy can be leveraged as described herein by harvesting cells from a surface or suspended cells and allowing them to replenish from beneath.

When different reactors are used for proliferation and differentiation, then the cells are transferred from one reactor to the other, with the different culture media in the different reactors. It should be appreciated that the different reactors may be the same type of reactor, but merely with a different culture media inside of it, though different styles or sizes of reactors are also contemplated.

In some cases, it may be advantageous to do one type of culture for proliferation and a different type of culture for differentiation. For example, if may be useful to conduct proliferation in single cell suspension, but to conduct differentiation using an encapsulation matrix, such as the ones described elsewhere herein.

After the adipose cells 12 have accumulated sufficient lipid and a desired amount (or level or number) of adipose cells 12 are generated, the culture is ended, and the lipid-laden adipose cells 12 are harvested according to a block 16. In some embodiments, the block 16 may include detaching the adipose cells 12 from a substrate and draining the adipose cells of non-cell liquid.

Cells can be harvested via the same techniques used to separate cells from culture media between the various stages.

As discussed elsewhere, “skimming” floating cells from the surface is one particularly promising approach to harvesting cells, given that it is an automatic indicator of buoyancy. “Floating” as used herein may be used interchangeably with “non-adhered” unless discussed otherwise. Other methods of collecting non-adhered cells may comprise collecting non-clustered floating cells, collecting only non-adhered cells, and/or not collecting adhered cells.

Without limit, conventional cell harvesting techniques may be used, such as those disclosed in Dryden et al., “Technical and Economic Considerations of Cell Culture Harvest and Clarification Technologies”, Biochem Eng J, Vol. 167, 107892, which is incorporated herein in its entirety by reference. While Applicant believes that some of the cell harvesting techniques described in this application are inventive, there are other inventive concepts which are usable with conventional cell harvesting techniques.

Prior to molding the harvested cells in the next portion of certain methods, some post-processing may be required. For example, the harvested cells might be rinsed to remove/minimize/dilute undesired culture media components. Rinsing can be done by way of counter-flow centrifugation or conventional filtration. As another example, cells can be clarified by removing undesirable components, which are not removed by rinsing. This is a process that is regularly used in the pharmaceutical arts, but it is not conventionally used in cellular agriculture. In one specific example, clarification of cells to remove cell debris or excess nucleic acids is expressly contemplated. Other methods such as flocculation via pH or the use of polymers such as chitosan which can induce flocculation are contemplated.

It is contemplated that the harvested adipose cells 12 themselves may be an end product, so the system and method can end there in certain circumstances. The harvested adipose cells 12 can be provided to customers for incorporation into products, including those outlined elsewhere herein.

At a next block 18, the harvested adipose cells 12 may be aggregated, for instance into a 3D mold (e.g., a 3D printed mold) having a desired 3D shape to generate the 3D adipose tissue 10.

The 3D mold can be a permanent and/or reusable mold or it can be single-use.

In the case of permanent and reusable molds, the important properties are durability, ability to be sterilized, and the like. Skilled artisans will understand that a variety of materials, from polydimethyl siloxane to various metals could be suitable for molds. The specific mold material for permanent and reusable molds is not intended to be limiting.

In the case of single-use molds, there are a variety of features that take advantage of the methods disclosed herein. In some cases, the single-use mold can be made of the same material as the materials identified elsewhere herein as hydrogel materials for use in the cell aggregation step.

In cases where the single-use mold is edible, the entire structure of the single-use mold and the cultured adipose tissue that is molded inside of it can be utilized for inclusion in the various products identified herein. For example, a large chunk of cultured adipose tissue and the 3D mold in which it was made can be chopped into smaller pieces for incorporating into a food product. In these cases, it is important for the material of the 3D mold to be able to withstand the temperatures that are associated with cooking without degrading or forming any undesirable reaction products. The material of the 3D mold can be stable or can simply change phase (e.g., similar to the rendering of fat) when exposed to temperatures of at least 40° C., at least 45° C., at least 50° C., at least 55° C., at least 60° C., at least 65° C., at least 70° C., at least 75° C., at least 85° C., at least 90° C., at least 100° C., at least 105° C., at least 125° C., or at least 150° C. or at temperatures at ranges of from 40to 150° C., from 50 to 125° C., from 60 to 105° C., or from 70 to 100° C. In these cases, the 3D mold material may be stable or generally stable in water, because so many food and cooking environments are aqueous. To the extent that it is not explicitly stated elsewhere, these properties may also apply to the binder/hydrogel material. A thickening agent or thickener may be a suitable binder/hydrogel.

As far as specific shapes are concerned, any practical shape can be suitable for use, though some may be preferred over others. For example, in some cases, generally regular spherical shapes may be preferred, while in other cases, irregular shapes may be desired. In some cases, the 3D shape may be elongated strips.

In some cases, where the desired shape is very small, it may be impossible or impractical to make molds of the desired shape. In these cases, it may be useful to make a larger sample that is carved into smaller pieces. For example. if a customer desires a host of small fat “flakes” for incorporating into a food product, it is much easier to make a large piece of cultured adipose tissue and then manipulate that large piece to make smaller flakes. Larger pieces can be cut, sliced, chopped, minced, ground, or other conventional way to manipulate animal-fat-like products.

In some cases, no mold is used at all. In these cases, the aggregated material may be formed by way of an extrusion or printing process.

In the case of extrusion, the cells and hydrogel material may be co-extruded to form a linear aggregate. The linear aggregate can be cut into pieces to form individual pieces of cultured adipose tissue. The linear aggregate can be solid or hollow, depending on the specific desired application.

Without wishing to be bound by any particular theory, it is believed that candy making processes, where ropes of extruded or rolled candy is cut into smaller pieces might be one example of a suitable process to mimic for an extrusion process for use with the present invention. Some extruded metal or rubber or plastic processes may be similarly applicable.

In the case of printing, the cells and binder/hydrogel material can be 3D printed into a desired shape.

In some embodiments, aggregating as shown in block 18 may involve embedding the harvested adipose cells 12 in a hydrogel or mixing with a binder in a 3D mold. Suitable hydrogels or binders include, but are not limited to, food safe compounds such as alginate, cellulose, gelatin, starch (e.g., tapioca), hyaluronic acid, fibrin, carrageenan, cornstarch, gellan gum, lecithin, maltodextrin, methylcellulose, carboxymethylcellulose, cellulose gum, hydroxypropyl cellulose, hydroxypropyl methylcellulose tragacanth, karaya, acacia, ghatti, beta-glucan, psyllium husk, inulin, chitosan, curdlan, dextran, potato starch, tapioca/arrowroot starch, hyaluronic acid, fibrin, carrageenan, guar gum, inulin, konjac, oat bran, pectin, locust bean gum, xanthan gum, soy protein, wheat protein, pea protein, chickpea protein, almond protein, rice protein, hemp protein, quinoa protein, sunflower protein, potato protein, algae protein, yeast protein, bacterial protein, corn protein, silk protein, cellulose derivatives, pullulan, hydrolysates of any of the aforementioned proteins, and combinations thereof. In some embodiments, the hydrogel or binder is alginate which is a material used as a fat replacer in the food industry. For instance, block 18 may include mixing the harvested and drained adipose cells 12 with an alginate solution at a specified volumetric ratio in the 3D mold. In one specific embodiment, a slow gelling alginate solution may be prepared by adding calcium carbonate and glucono delta-lactone (GDL) powders to an alginate solution, and the slow gelling alginate solution may be combined with the harvested and drained adipose tissue at a 1:1 volumetric ratio in a 3D printed mold (see Example 3). For other binders/hydrogel materials in this list, the process is typically a heat-driven process, where water and the binder/hydrogel ingredient are heated, fat cells are mixed in, and the mixture is cooled. As a specific example, agar/agarose is heated to liquify it (e.g., 85-100° C.), then cooled (e.g., 40-65 °C)and mixed with adipocytes. After mixing, the solution is cooled to form a solid piece of macroscale, structured fat.

In some aspects, aggregating as shown in block 18 may involve cross-linking the harvested adipose cells 12 in a 3D mold. The cross-linking may be carried out using a suitable protein-protein cross-linking enzyme such as, but not limited to, transglutaminase, a tyrosinase, a peroxidase, a laccase, a sortase, a subtilisin, and lysyl oxidase. In some aspects, cross-linking the harvested adipose cells includes enzymatically cross-linking the harvested adipose cells using transglutaminase. For example, cross-linking the harvested adipose cells may involve mixing a solution of transglutaminase with the harvested adipose cells at a specified volumetric ratio in a 3D mold (see Example 3). The block 18 may further include adding helper proteins during the cross-linking. In some embodiments, the helper proteins (which may simply be referred to as “proteins”) may be selected from casein and gelatin. Chemical crosslinking can also be used when the reactants or catalysts are food safe, such as EDC-NHS reactions between acid and amine groups. Photochemical crosslinking can also be utilized where photosensitizers are food safe. Without wishing to be bound by any particular theory, it is believed that using 6% or higher of soy protein isolate and 5-25 enzyme activity units/gram of protein substrate of transglutaminase is one suitable starting point for these crosslinking conditions. Higher soy protein isolate and/or transglutaminase content can lead to higher strength within the gel. However, the maximum strength is not necessarily desirable, because it would not mimic the mouthfeel of fat. In some cases, a mixture of pea protein isolate (19.6% w/w), transglutaminase (0.7% w/w), and NaCl (1% w/w) with a pH of 6.5 is incubated at 50° C. to provide a crosslinked protein matrix that is often referred to as a plant based meat. Adipocytes can be mixed into this composition before curing to form a hybrid product of culture adipose tissue and plant based meat.

In some cases, the crosslinking may be dependent on one or more cofactors or metals. Exemplary cofactors or metals may include calcium, copper, and heme. In some cases, the protein is mixed in with the binder/hydrogel and crosslinked (with the transglutaminase, for example). This may be necessary to form a gel.

In some cases, canola oil can be mixed with <30% (w/w) of fully hydrogenated canola oil at 65° C., hot-emulsified with a soy protein suspension (8%, w/w) at a lipid content of 70% (w/w) using a high-shear disperser, and cooled to 37° C. The concentrated, emulsified fat crystal networks can then incubate with transglutaminase for 1 hr to induce protein crosslinking.

Alternatively, other types of cross-linkers may be used for adipose cell aggregation such as, but not limited to, polymers functionalized with aldehyde groups, genipin, phenolic compounds, and combinations thereof. Suitable polymers functionalized with aldehyde groups include, but are not limited to, periodate oxidized pectin, dextran, chitosan, Arabic gum, sucrose, raffinose, stachyose, cyclodextrin, and starch. Suitable phenolic compounds include, but are not limited to, caffeic acid, chlorogenic acid, caftaric acid, quercetin, and rutin derived from plants such as grapes and coffee.

In some cases, aggregation of the cells or the combination of both the aggregation and harvesting of non-adhered adipose cells comprises flocculation. As used herein, “flocculation” and grammatical equivalents may refer to the aggregation of unstable and small particles through surface charge neutralization, electrostatic patching and/or bridging after addition of flocculants (or agents that make fine and sub-fine solids or colloids suspended in the solution form large loose flocs through bridging thus achieving solid-liquid separation). Flocculation may be performed by one or more of several techniques, including auto-flocculation, bio-flocculation, chemical flocculation, particle-based flocculation, and electrochemical flocculation. Various flocculation techniques have been reviewed by Matter, et al (2019, Applied Sciences). This is a method with high potential as it might be used to harvest cells from the bioreactor at low cost.

Auto-flocculation may be performed at acidic pH (for example, a pH of 4.0). Auto-flocculation may be performed at alkaline pH (for example, a pH of 10.4, 11.0, 1.5, 11.6, 12.0, or 12.5). Auto-flocculation may be performed over several days (for example, 16 days).

Chemical flocculation may be performed with inorganic flocculants such as aluminum sulfate, calcium oxide, iron(III) chloride, and magnesium hydroxide. Chemical flocculation may be performed with organic flocculants such as inulin, starches, chitosan, tannins, alginates, and lysines. Flocculation may preferably be performed with food-safe flocculants such as chitosan, cellulose, lignin and/or other long polymers.

Particle-based flocculation may be performed using aminoclay-based nanoparticles such as 3-aminopropyltriethoxysilane (APTES) conjugated to magnesium and aminoclays conjugated to aluminum, titanium oxide, and humic acid. Particle-based flocculation may be performed using magnetic particles including ferrous oxide nanoparticles and composites.

Coagulation of cells may be an additional or alternative means of cell harvesting and/or aggregation that is similar to flocculation wherein cells aggregate or clump.

The adipose cells 12 or the adipose tissue 10 may be supplemented at various stages to tune the sensorial characteristics (e.g., texture, color, and flavor) and/or the nutritional attributes of the cultured adipose tissue 10. For example, supplementation with additives such as, but not limited to, flavorants, colorants, texturizers, vitamins, minerals, amino acids, proteins/peptides, oils, whole foods, and fatty acids is also encompassed by the present disclosure. In some embodiments, tunable control of fat nutrition and health may be implemented. The fatty acid composition of the cultured adipose tissue may be tailored via cell feeding strategies, such as by supplementing fatty acids into the culture media during in vitro culture. Genetic interventions may also serve to bolster the nutrition of the cultured adipose tissue 10. For example, in one embodiment, omega 3 desaturases may be expressed or pathways to produce lipophilic nutrients (e.g., beta carotene, vitamin A) may be activated in the adipose cells 12. This may be beneficial to the consumer as certain nutrients are more bioavailable when consumed in food versus a micronutrient supplement. Additionally, the texture of the cultured fat may be tunable based on variables such as the hydrogel/binder (e.g., alginate) concentration, cross-linker levels, and the inclusion of helper proteins (e.g., casein, gelatin, etc.) during cross-linking. In one embodiment, the cultured adipose cells 12 may be supplemented with methylated branched fatty acids to impart a “mutton” flavor in the cultured adipose cells 12. One specific methylated branched fatty acid is 4-methyloctanoic acid. Additionally, the relative extracellular matrix production and fat production levels may be optimized pending the texture, taste, and/or nutritional outcomes desired.

In one particular case involving fish cells, the starting material can be fish muscle cells, given that fat cells are generally harder to acquire from certain fish species known to be very lean. It has also been shown that certain fish muscle cells can self-immortalize, which is advantageous for large scale production. It is possible to induce lipid uptake in skeletal muscle cells via fatty acid/lipid supplementation, forming fatty muscle cells. Fatty acid/lipid supplementation may also induce transdifferentiation of skeletal muscle cells into adipocytes, or adipocyte-like cells. Genetic approaches may also be used to transdifferentiate skeletal muscle cells, such as via the activation or insertion of PPARγ. Without intending to alter the definition provided above, these fatty muscle cells can be used wherever adipocytes or fat cells are referenced herein.

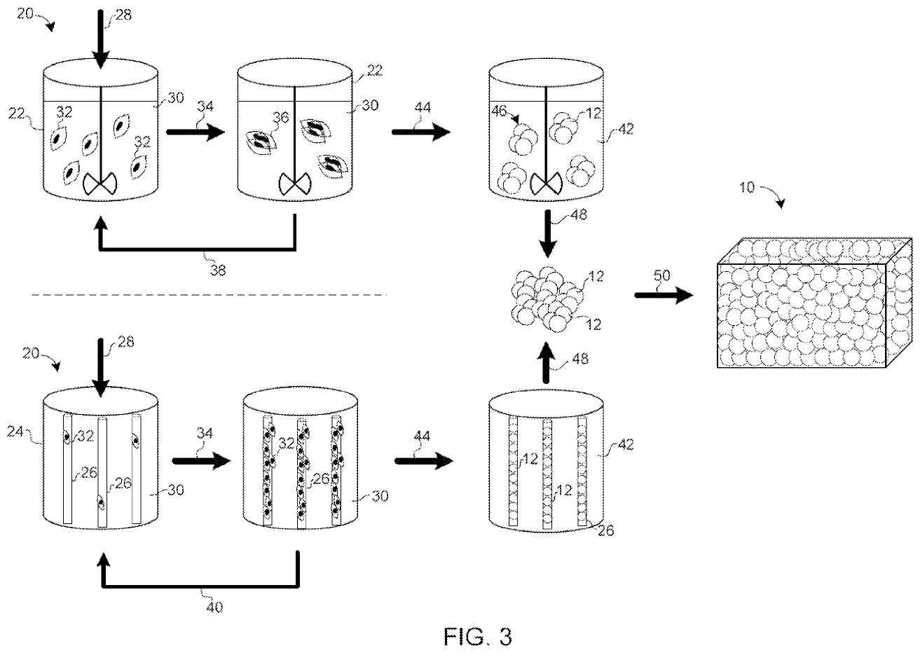

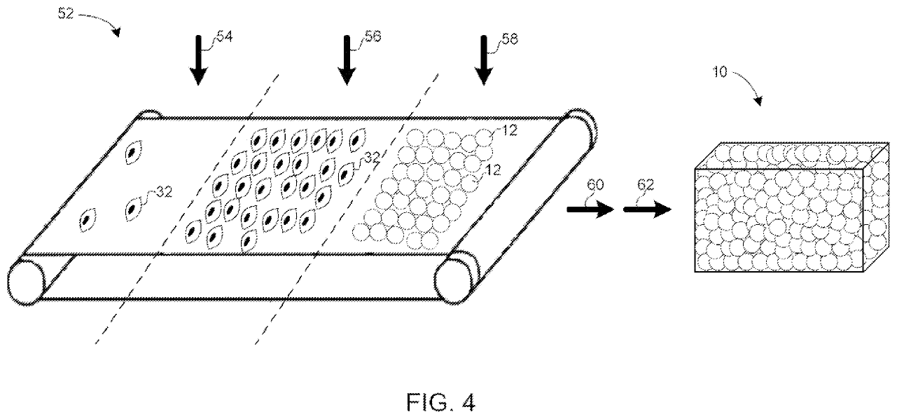

FIG. 3 shows scalable processes for the mass production of the cultured adipose tissue 10. The processes may be carried out in a bioreactor 20, such as a stirred suspension tank bioreactor 22 (top) or a hollow fiber bioreactor 24 having hollow fiber membranes 26 (bottom). Other types of bioreactors apparent to those skilled in the art may also be used and are within the scope of the present disclosure such as, but not limited to, rotating wall vessel bioreactors (RWVBs) and packed bed bioreactors. Production of the adipose tissue 10 in the bioreactor 20 may involve seeding 28 a first culture media 30 (adipogenic induction media) in the bioreactor 20 with adipogenic precursor cells 32. The adipogenic precursor cells 32 may then proliferate 34 to confluency (or to a desired coverage/number of cells on a surface or in suspension) in the bioreactor 20. In some embodiments, the adipogenic precursor cells 32 may form small aggregates or spheroids 36 as they proliferate (see FIG. 3, top). The spheroids 36 may be dissociated 38 into single adipogenic precursor cells 32 and allowed to proliferate 34 further (see FIG. 3, top). In the hollow fiber reactor 24, the adipogenic precursor cells 32 may proliferate on the surface of the hollow fiber membranes 26 (see FIG. 4, bottom). In this case, the adipogenic precursor cells 32 may be detached 40 from the hollow fiber membranes 26, and the detached adipogenic precursor cells 32 may be used to re-seed the media 30 for further proliferation 34.

As the first culture media 30 is changed to a second culture media 42 (the lipid accumulation media), the cells may accumulate lipids and differentiate 44 into adipose cells 12. The adipose cells 12 may grow separately or in small clusters 46 (see FIG. 3, top). In the hollow fiber bioreactor 24, the adipose cells 12 may develop on the surface of the hollow fiber membranes 26. In some embodiments, a single culture medium may be used for both proliferation 34 and differentiation 44. After the adipose cells 12 have grown and accumulated sufficient lipid, the adipose cells 12 may be harvested 48. In the hollow fiber bioreactor 24, the harvesting may involve detaching the adipose cells 12 from the hollow fiber membranes 26. The harvested adipose cells 12 may then be aggregated 50 in a 3D mold to provide the cultured adipose tissue 10. As explained above, suitable methods for binding and aggregating 50 the adipose cells 12 include cross-linking (e.g., enzymatic cross-linking with transglutaminase), as well as embedding the adipose cells 12 in hydrogels such as alginate.

The culturing process of the present disclosure may be compatible with two dimensional (2D) culture strategies. In some embodiments, the adipose cells 12 may be cultured in thin layers on a 2D substrate such as culture plates, and then aggregated into the 3D adipose tissue 10 according to the above-described procedures. For instance, the adipogenic precursor cells 32 may be grown to confluency (or to a desired coverage/number of cells on a surface or in suspension) and differentiated into the adipose cells 12 on the 2D substrate. Harvesting or collecting the adipose cells 12 from the 2D substrate followed by aggregating the harvested adipose cells 12 may provide the cultured adipose tissue 10. In some embodiments, the 2D substrate may by edible and incorporated into the final food product, such that the adipose cells 12 do not need to be detached from the 2D substrate. In some embodiments cells may grow on the 2D substrate and eventually become non-adherent and the adherent and non-adherent cells may be harvested and/or aggregated separately and/or used for distinct purposes as detailed herein.

In a continuous, assembly line-like process (or “assembly line process”) for the mass production of the cultured adipose tissue 10, the 2D substrate may be a conveyor belt 52 (see FIG. 4). The continuous production process may involve seeding 54 the adipogenic precursor cells 32 onto the conveyor belt 52 having a culture media thereon. The adipogenic precursor cells 32 may then proliferate 56 to confluency (or to a desired coverage/number of cells on a surface or in suspension) on the conveyor belt 52. Changing the culture media to lipid accumulation media may allow the adipogenic precursor cells 32 to accumulate lipid and differentiate 58 into the adipose cells 12. Alternatively, a single culture medium may be used for both proliferation 56 and differentiation 58. The adipose cells 12 may be harvested 60 by detachment from the conveyor belt 52, and then aggregated 62 according to the above-described procedures to provide the cultured adipose tissue 10.

The technology disclosed herein provides a novel and scalable approach to cultured fat generation. The present disclosure leverages large-scale cell proliferation and scale up technology to generate a required amount of in vitro adipose cells, after which the cells are aggregated or packed into a solid 3D construct on the macroscale. The adipose cells are cultured in thin layers (2D culture) or in bioreactors with easy access to the culture media, followed by aggregation into macroscale 3D tissues after sufficient adipocyte maturation. The aggregation of adipocytes or adipocyte clusters recapitulates native fat tissue from a sensory perspective as adipose tissue in vivo is largely a dense aggregation of lipid filled adipocytes with a sparse extracellular matrix. Furthermore, the compatibility of the adipose tissue production method with 2D culture strategies allows for a continuous production process with a conveyor belt assembly line approach.

Additionally, the method of the present disclosure produces bulk cultured adipose tissue in a way that circumvents the mass transport limitations associated with directly culturing or engineering large 3D tissues. Aggregation at the end of cell culture removes the need for nutrient delivery to the adipose cells via vascularization or an elaborate tissue perforation system. This is because, for food applications, the cultured adipose cells do not need to stay alive once formed into the final edible tissue. This is analogous to meat production in conventional animal agriculture where muscle and fat cells gradually cease to be viable after slaughter. In contrast, for medical applications, cells in 3D tissues may be expected to remain viable to be used for implantation into the body or for testing in an in vitro tissue model. Accordingly, the adipose tissue production method of the present disclosure is less costly than other methods that rely on complex perfusion and mixing systems to distribute nutrients during cell growth. In some embodiments, the food product described herein is produced without vascularization or perfusion.

According to the methods of the present disclosure, monocultures of adipocytes and pre-adipocytes may be sufficient for the production of large fat droplets without the need for supporting cell types. Standard cell culture conditions are sufficient for the type of adipocyte culture outlined in this disclosure, and no specific coatings on tissue culture plastics were required to achieve desired adipocyte growth and development. Furthermore, the pre-adipocytes and adipocytes of various livestock species may be grown in serum-free culture media according to the present disclosure, thereby eliminating a major obstacle in in vitro fat culture. These advantages further help reduce production costs. Co-cultures can also be considered for enhanced fat outcomes, such as the use of fibroblasts or muscle cells in the cultures, such as to increase the quality of the fat products or to alter the texture and composition.

Applicant has also observed that a large subpopulation of the cultured adipocytes adhere strongly to tissue culture plates and do not float away, avoiding issues of lift-off of adherent adipocytes in vitro due to increasing buoyancy as the adipocytes become fatty. The 2D culture systems disclosed herein self-sort for adherent cell populations, which may provide for adherent and non-adherent cell populations that may be used for separate downstream applications. Applicant has also developed techniques for dealing with or using the non-adherent cell populations, as described elsewhere herein.

The systems and methods described herein can include various sensing and control systems to facilitate superior control over the processing/harvesting process. At a general level, the systems and methods include a measurement component, which observes a property of an individual cell or a group of cells, and an assessment component, which assesses the measurement to make a processing/harvesting decision to select an appropriate time for processing/harvesting the cell or group of cells. As used herein, the term processing refers generally to a decision to move a cell or a population of cells to a different processing stage. These processing steps can in some cases be related to developmental differentiation (i.e., the process of becoming an adipose cell). As used herein, the term harvesting generally refers to a final processing step for adipose cells, after which those cells are aggregated as described herein or otherwise further processed in ways that do not materially alter the cells from their state at harvest.

In some cases, the processing/harvesting decisions can be based on monitoring of individual cells. Without wishing to be bound by any particular theory, it is believed that analytical (e.g., optical, etc.) techniques that interrogate individual cells can provide meaningful distinctions between adipose cells that are ripe for processing/harvesting versus adipose cells that are not yet ready. For example, individual optical interrogation of cells revealing underlying lipid accumulation can be performed, followed by comparing that interrogation to a benchmark threshold for cells known to have a desired lipid accumulation (or other measurable property). These techniques can include active techniques, where the property or properties of the cells are directly measured, and more passive techniques, where the property or properties of the cells are more indirectly observed.

In some cases, the processing/harvesting decisions can be based on monitoring of populations of cells. Without wishing to be bound by any particular theory, it is believed that analytical (e.g., optical, electrical, etc.) techniques that interrogate populations of cells can provide meaningful distinctions between the ripeness of those populations for processing/harvesting. For example, populations of cells can have optical measurements taken (e.g., absorption, scattering measurements, etc.) or electrical measurements taken (e.g., impedance, capacitance, etc.), which can represent important meaningful properties of the population of cells. These techniques can include active techniques, where the property or properties of the population of cells are measured directly, and more passive techniques, where the property or properties of the population of cells are more indirectly observed. Lipid accumulation may be a suitable determinant for monitoring.

The processing/harvesting decisions can be based on cell growth. In some cases, growth can be determined in terms of growth of individual cells (i.e., cell size and/or mass and/or density). In some cases, growth can be determined in terms of growth of the number of cells (i.e., cell count).

The processing/harvesting decisions can be based on a degree of cell differentiation. As described elsewhere herein, the system and method can include steps relating to differentiation of certain cell types into adipose cells and/or precursors of adipose cells.

The monitoring can be user-originated, such that a user can specifically request that the system or method observe a given cell or a given population of cells at a specific time.

The monitoring can be automated, such that the monitoring occurs at a predetermined time or that a computer program selected a time based on a given set of criteria. Skilled artisans in the automation arts will recognize a host of options for automating the monitoring of the individual cells or populations of cells.

In some cases, the monitoring involves a destructive sampling process, where one or more cells are removed from the system or method in order to make a representative measurement.

For manual destructive sampling processes, traditional sampling techniques can be used, such as physical removal of cells or populations of cells. A user can arbitrarily select the cells for sampling or the user can be directed in some fashion to select specific cells and/or a specific location. In cases where specific direction is given, the system can include labels that direct the user to a proper sampling location. In some cases, these labels can be digital labels or they can be printed labels.

For automatic destructive sampling processes, a variety of sampling complexities can be pursued. On the simpler end of the spectrum, automated sampling can involve routinely retrieving cells from a specific location in the system/method and simply acquiring whatever sample happens to be occupying that location at the given time. This is a good bulk sampling technique to be used in cases where the overall number of cells required for sampling is adequately small when compared with the overall cell population size. On the more complicated end of the spectrum, automated sampling can involve complex decision trees and/or machine-learning-derived algorithms for selecting cells or populations of cells for interrogation. Examples of suitable selection algorithms include, but are not limited to, random sampling techniques, weighted sampling techniques, and the like.

In some cases, the monitoring involves a non-destructive sampling process, where the cells are not disturbed to a degree that their growth, differentiation, and/or proliferation remain generally unaltered.

For manual, non-destructive sampling processes, the user can manually move an analytical device into a location for interrogating a cell or population of cells. A user can arbitrarily select the cells for interrogation or the user can be directed in some fashion to select specific cells and/or a specific location. As with the destructive techniques, the systems and methods can utilize labels to direct a user to a proper location. Such labels can be digital or analog.

In some cases, the monitoring is performed on adhered cells. In these cases, the monitoring systems are configured to interrogate a location where the substrate is positioned during processing. Because the systems and methods are typically deployed in a reproducible fashion, the substrates will typically be located in predictable locations.

In some cases, there is no need to examine adhered cells for harvesting decisions, because their adherence is a reflection of inadequate fat content. In other words, if the adherence is tuned to a given general adherence, then detachment can serve as the sign that the cell is ready for harvest.

In other cases, the monitoring can be performed on non-adhered cells. Those aspects of the present disclosure are described in greater detailed elsewhere herein where “floating” cells are discussed.

In one specific example, the monitoring is a specific monitoring of lipid droplet size within a cell or within a population of cells. Without wishing to be bound by any particular theory, it is believed that lipid droplet size is an important factor in determining when an adipose cell is ready for harvesting. This lipid droplet size is optically visible and can be interrogated by known optical methods. It should be appreciated that the lipid droplet size monitoring can also be used to select for undesirable cells, in a similar fashion to the way that it selects for desirable cells.

In some cases, the monitoring can include monitoring the culture media. This analyzing of the culture media can be used for making harvesting decisions or can inform control of other aspects of the method.

In one case, the culture media can be monitored to determine a concentration of ammonia. The concentration of ammonia can be used as a feedback for manual or automated control of the system.

In certain cases, the ammonia can be extracted and processed into an end product. For example, ammonia can be extracted and processed into fertilizer using methods known in the art.

In one case, the culture media can be monitored to determine a concentration of lactic acid in the culture media. The concentration of lactic acid can be used as a feedback for manual or automated control of the system.

In general, waste products that are not useful as recycled source materials in the system and method described herein can be monitored and removed from the system. If those waste products are capable of being transformed into a useful product, then the system can include those facilities.

In certain cases, the lactic acid can be extracted and processed into an end product. For example, lactic acid can be extracted and processed into poly(lactic acid) using methods known in the art.

In one case, harvesting decisions can be phenotypic.

The harvesting decisions can be supplemented with various feedback loops, relaying information about the process as it is underway.

In certain cases, the secretome of the cells can be used to make a harvesting decision. In some cases, the level of adipokine surrounding the cells can be used as a measure of readiness for harvest. Similarly, the level of adiponectin could be utilized for harvesting decisions.

In some cases, the systems and methods described herein are particularly useful for processing cells that detach from the substrate during the process of differentiating into fat cells (herein referred to as “floating” or “non-adhered” cells). The systems and methods of this disclosure provide approaches to collecting (or “harvesting” or “aggregating”) and utilizing these floating cells, which have traditionally been discarded as waste. By making productive use of these previously-wasted products, much higher efficiency is achieved by virtue of not wasting this large population of cells.

In one case, the surface of a growth medium reservoir can be monitored to determine a concentration of floating cells. In other words, a liquid-air interface is monitored. If the concentration of floating cells exceeds a predetermined threshold, then the systems and methods can harvest the floating cells. These harvested floating cells can be processed in the fashion described elsewhere herein.

In another case, an interface between two liquids having different densities can be monitored to determine a concentration of cells at the interface. In other words, a liquid-liquid interface is monitored.

In yet another case, multiple interfaces between multiple liquids having different densities can be monitored to determined concentrations of cells at the multiple interfaces. In other words, multiple liquid-liquid interfaces are monitored.

In the case where multiple different density gradients are deployed and/or where multiple different populations of floating cells are collected, these cells can be divided into quality grades based on one or more properties.

In some cases, cells having a lower density can be assigned a higher grade.

In some cases, cells that remain adhered to a substrate for a shorter length of time are assigned a higher quality grade. In some cases, cells that remain adhered to a substrate for a longer length of time are assigned the higher quality grade. It should be appreciated that some applications may have a preference for cells that rapidly loser their adherence versus cells that slowly lose their adherence and vice versa. Without wishing to be bound by any particular theory, the systems and methods described herein provide a platform for assigning quality grades to a given cell.

In some cases, the floating cells can be delivered to a chromatographic system and/or a microfluidics system and/or a cell sorting system and/or a cell picker, as will be understood by those having ordinary skill in the cell manipulation arts. A cell skimmer is one particularly useful device for removing cells from the surface.

In cases where the floating cells are directed to the microfluidics system, the microfluidics system can be tailored specifically for handling floating cells described herein. One example of tailoring the microfluidics system for floating cells is to adjust the density of the carrier fluid prior to entry into the microfluidics system, such that the cells are neutral buoyance and are no longer “floating” in the medium.

In cases where the floating cells are directed to the chromatography system, a specific stationary phase and mobile phase may be selected for a desired result. For instance, if size selection is an important criterion, then a stationary phase that has porosity that provides differentiated retention properties based on size could be useful. As another example, if surface chemistry of the adipose cells is an important criterion, then a stationary and/or mobile phase could be selected based on those desirable surface chemistries. In one specific example, certain cells may be more amenable to embedding into a hydrogel if they have a specific surface chemistry, so a chromatography system could be used to isolate cells that are more or less amenable to embedding into the hydrogel. Similarly, in another specific example, certain cells may be more amenable to direct cross-linking to other cells if they have a specific surface chemistry, so a chromatography system could be used to isolate cells that are more or less amenable to direct cross-linking with one another.

In cases where the floating cells are directed to a cell sorting system, the cell sorting system can be used to differentiate and categorize the cells based on measurable properties, as would be appreciated by a skilled artisan. Applicant does not intend to provide specific inventive contribution with respect to the cell sorting itself, but rather integrates and utilizes the understood concept of cell sorting into inventive concepts that form a part of the broader disclosure described herein.

In some cases, the cells are sorted through multiple different centrifuges. In that case, the cells that make it through multiple centrifuges will have different properties from those that do not and those different categories of cells can be divided into grades, in a similar fashion as USDA grading. The cells could be graded as superior or inferior relative to one another.

In some cases, the cell sorting can involve the use of a density gradient. In those cases, the different densities can allow harvesting of cells having different buoyancies. In those cases, the different buoyancies can also be used to grade the cells.

In cases where the cells are graded, some cells that do not meet certain grading threshold or certain quality standards (i.e., cells that do not have enough fat in them) can be returned to the system at the relevant stage in the process.

While this section focuses on techniques that are applicable to floating cells, similar techniques may be applicable to sedimented cells, as would be recognized by those having ordinary skill in the art. For example, rather than monitoring a surface interface, the bottom of a reaction tank could be monitored for sedimented cells. Sedimented cells may be preferred for certain applications and those sedimented cells can be collected and further processed for those applications. However, given the focus of this application as it relates to adipose tissue, which is generally buoyant in aqueous environments such as growth cultures, the sedimented cells are typically waste products and can be processed as such.

In some cases, sedimented cells can be isolated and further processed as biomass.

In some cases, the sedimented cells can be recycled for raw materials and reintroduced into the system and/or method at various points in the system/process. For example, sedimented cells can be harvested and have components for culture media extracted from them.

In some cases, the sedimented cells are processed for other uses. For example, sedimented cells might be suitable filler material in animal feedstock. As another example, sedimented cells might be suitable for use as a starting material for a further culture. Without wishing to be bound by any particular theory, it is believed that the systems and methods described herein may provide useful sorting and/or isolation of relevant cell populations, such as the sedimented cells described herein. These sedimented cells are artificially selected by virtue of the processing decisions made. These artificial selections can imbue the cell populations with desirable properties (e.g., density can be desirable for certain applications that are not trying to make adipose tissue) and further cultures can grow and expand those populations of cells to have larger populations that are enriched in those desirable properties.

In some cases, the sedimented cells themselves may be usable as a single cell protein for growth of bacteria or fungus. The cell itself serves as the protein source.

One disadvantage of sedimented cells is that they are typically very high in nucleic acid content. Therefore, in some cases, nucleic acid content is reduced prior to harvesting non-adhered adipose cells. In some cases, the sedimented cells can have nucleic acid content reduced before further use. However, the cells may still be useful without reducing the nucleic acid content, for example, as livestock feed. In some cases, the sedimented cells, with or without reducing the nucleic acid content, can be mixed into one or more of the food products described herein for the purpose of adding more of a “meat” flavor. Reducing nucleic acid content may comprise “rinsing” cells as understood in the art and discussed herein elsewhere.

From a practical standpoint, all of the techniques that are applicable above with respect to the floating cells are applicable here, but with a reversal of the buoyancy impact. Rather than adjusting the density of the liquid down to provide neutral buoyancy (as in the case of floating cells), the density of the liquid would be adjusted up.

Any of the proposed genetic modifications discussed above are also applicable as additives, so long as the science does not render it impossible. For example, the carotenoids for adding color can simply be added to the cells as supplements. As another example, rather than genetically modifying cells to express more fatty acids, those fatty acids can be added to the cells via supplementing. The same is true for myoglobin and hemoglobin. In some cases, dyes may be added to the cells and/or binder/hydrogel in order to provide a more aesthetically pleasing appearance.

In certain cases, the cultured adipose tissue may be particularly useful for introducing lipid-soluble nutrient into subjects. For example, a lipid-soluble nutrient that is challenging for uptake in humans can be supplemented into the cells here and the resulting cultured adipose tissue may enable greater uptake of those lipid-soluble nutrients than if they were administered outside of the cultured adipose tissue.

Other additives that add no proven nutritional or therapeutic value are also contemplated. In some cases, additives can be purely aesthetic. For example, one additive could be gold nanoparticles, which could be added at any stage of the process and taken up into the cells, thereby producing “gold fat”. As another example, various materials from the nutraceutical industry can be incorporated into the cells at various stages of the process, including but not limited to silver compositions which are believed by some to have antimicrobial properties.

In some cases, the systems and methods described herein can provide advantageous ability to administer growth factors to the cells, in the interest of providing them with more optimal receipt and uptake of the growth factors.

Certain growth factors may advantageously be administered to cells for the purpose of aiding and/or enhancing their development in the systems and methods disclosed herein.

In some cases, some of the growth factors can be incorporated into the hydrogel or binder in which harvested adipose cells 12 are aggregated.

Similarly, additives can be incorporated into the hydrogel or binder in which harvested adipose cells 12 are aggregated. These additives can include, but are not limited to, flavorants, colorants, texturizers, vitamins, minerals, amino acids, proteins/peptides, and fatty acids. These additives can be added to the hydrogel or binder in amounts that are tuned to the desired outcome.

Growth factors that are suitable for use as additives include, but are not limited to, fibroblast growth factor 2, transforming growth factor beta 3, insulin-like growth factor, or the like. Growth factors may be fish-specific growth factors.

Without wishing to be bound by any particular theory, it is believed that cultured cells are generally deficient in oleic acid, linoleic acid, or arachidonic acid. These can all be additives in the present disclosure, thereby obviating the deficiency.

Alternative or additional additives include, but are not limited to, flavorants, colorants, texturizers, vitamins, minerals, amino acids, proteins/peptides, whole foods, oils, and fatty acids. Particular fatty acids, such as linoleic acid, arachidonic acid, alpha-linolenic acid, eicosapentaenoic acid, and docosahexaenoic acid, may be preferable but are not exclusive. Other additives may be selected from the group comprising soybean oil, coconut oil, palm oil, cocoa butter, shea butter, mango butter, olive oil, canola oil, sunflower oil, flaxseed oil, avocado oil, peanut oil, sesame oil, corn oil, vegetable oil, margarine, shortening, and vegetable ghee. Additives comprising fatty acids or oils may be hydrogenated at least partially (which includes wholly hydrogenated) or may be present in their oleogel, bigel, or emulgel form. Whole foods may be selected from the group comprising, but not limited to, tofu, tempeh, seitan, jackfruit, quinoa, chia seeds, bananas, oats, avocados, flax seeds, rice, tapioca/arrowroot, and potatoes.

One particularly useful additive for the present disclosure is a fatty acid. Without wishing to be bound by any particular theory, it is believed that cell lines that are generally lacking in the production of a desirable fatty acid can be supplemented in the development process to include more of the desirable fatty acid. Again, without wishing to be bound by any particular theory, it is believed that in vitro fat cells have historically been lacking in certain fatty acids.

The most abundantly missing fatty acid is linoleic acid (18:2), as mammals take this from the diet rather than synthesizing it themselves. There can also be a lack of arachidonic acid (20:4), as it is made from 18:2. The lack of 20:4 can be both good and bad, as it's been reported to be important for flavor but also a precursor molecule to pro-inflammatory compounds (eicosanoids).

In vitro mammalian cells are also typically lacking in omega 3 fatty acids as these are also dietary. These would be alpha-linolenic acid (18:3), eicosapentaenoic acid (EPA, 20:5), and docosahexaenoic acid (DHA, 22:6). Omega 3 s are generally less abundant in terrestrial (land) animals so a lack of them is not that detrimental. However, they can make up a significant proportion of fish lipids, so it may be important to have for fish adipocytes.

In a general sense, when multiple different fatty acids are supplemented, the supplementing concentrations can be chosen to provide desirable end concentrations.

In one specific example, soybean oil can be added to one or more of the stages of the system and method. Soybean oil has been shown to increase lipid drop size in in vitro adipose tissue. By adding soybean oil, a desired droplet size can be achieved more quickly, thereby enhancing efficiency of the overall process. Alternative or additional exemplary oils may include coconut oil, palm oil, cocoa butter, shea butter, mango butter, olive oil, canola oil, sunflower oil, flaxseed oil, avocado oil, peanut oil, sesame oil, corn oil, vegetable oil, margarine, shortening, and vegetable ghee. Oils may be partially or wholly hydrogenated and may be constituted in their oleogel, bigel, or emulgel form. Bigels and emulgels are promising for obtaining nutritionally highly improved meat products without affecting their sensory or physicochemical properties. A discussion of these advantages can be found in Dominguez, et al “Elevating meat products: Unleashing novel gel techniques for enhancing lipid profiles”, Meat Science, Volume 204, October 2023, which is incorporated herein in its entirety by reference.

In some cases, the additives mirror dietarily important additives as understood by those skilled in the dietary arts. For example, in the same way that medicine has distinguished recently between the “good” type of cholesterol and the “bad” type, the systems and methods described herein can be supplemented with the “good” type of cholesterol, thereby providing an end product with a desirable amount of this form of cholesterol.

In some cases, the additive might be present in the form of an uptake-enhancing additive for the explicit purpose of enhancing uptake of other additives. Without wishing to be bound by any particular theory, in a mice model, the uptake of triglycerides was enhanced by administering an agent that induced the release of a relevant lipase, which allowed the breakdown of the triglycerides for entry into the cells. A skilled artisan will recognize that certain co-administered agents may be required to be administered with a given additive in the interest of improving uptake of the additive. The uptake-enhancing additive can be an enzyme that modifies a different additive to make that different additive more suitable for uptake (e.g., a lipase with large lipids). The uptake-enhancing additive can be a permeability-enhancing agent, which enhances the permeability of the cell membrane of the adipose cells.

One specific avenue for introducing growth factors and/or agents and/or additive into the inventive compositions is by way of adding the growth factors and/or agents and/or additives into the first culture media or the second culture media at the time that those culture media are made and/or when those culture media are introduced into their respective tanks.

The culture media can be tailored for proliferation and differentiation, as well for other steps of the process. In addition, the various steps can use more than one culture media. For example, one culture media could be used for a first part of a proliferation process and the cells can be transferred to a different culture media for a later part of the proliferation process.

The culture media is typically comprised of a basal medium containing basic nutrients (sugars/carbohydrates, lipids/fatty acids, amino acids, vitamins, minerals, salts, water) combined with a growth factor or complex component containing proteins, peptides, hormones, or other bioactive compounds responsible for directing cell behavior. (e.g., to promote proliferation/cell survival, or to promote adipogenesis, or to suppress other cell pathways such as osteogenesis) Basal media will likely be best optimized for the specific adipogenic progenitor cell that is used but may be similar to or based off of existing formulations such as Dulbecco's modified eagle medium (DMEM), Ham's F12, Ham's MCDB 131, Iscove's modified Dulbecco's medium (IMDM).

Growth factors for adipogenic progenitor proliferation media formulations may include non-exhaustively: serum albumins, basic fibroblast growth factor (bFGF), epidermal growth factor (EGF), platelet derived growth factor (PDGF), fetuin, bone morphogenic proteins (BMPs, e.g., BMP4), Wnt proteins, leukemia inhibitor factor (LIF), hydrocortisone, testosterone, progesterone, estrogen, hypoxia inducible factors (HIFs), stem cell growth factor-β (SCGF-β), tumor necrosis factor alpha (TNFα), interleukin-1 beta (IL-1β) and other interleukins (ILs), transforming growth factor beta (TGFβ), insulin-like growth factor 1 (IGF-1), insulin, transferrin, myoglobin, vitronectin, truncated vitronectin peptides, laminin, laminin peptides (e.g., 511-E8), fibronectin, collagen, gelatin, trypsin inhibitors.

Growth factors specifically for adipogenic progenitor cell differentiation include non-exhaustively: Phospholipids, cholesterols (e.g., very low density lipoprotein, VLDL), fatty acids (e.g., oleic acid, linoleic acid), triacylglycerides (e.g., soybean oil, olive oil, rapeseed oil, fish oil), cyclodextrins (to solubilize non-polar ingredients), PPARγ agonists (e.g., rosiglitazone, pioglitazone, pristanic acid, phytanic acid, lutein, beta-carotene, all-trans retinoic acid, 9-cis retinoic acid), ascorbic acid, isobutylmethylxanthine (IBMX), dexamethasone, hydrocortisone, insulin, insulin-like growth factor 1 (IGF-1). In some cases, components that promote differentiation may overlap with ingredients in the basal medium formulation.

In some embodiments, the effects of more complex growth factors may be replicated more cost effectively using peptides or small molecules. Growth factors, in particular large protein growth factors, may also be modified to impart features such as improved thermal stability over time.

In certain embodiments, certain growth factors or media components can be excluded to reduce costs, should the cells be engineered to not require such components/be engineered to generate the components themselves from components or basic/simple nutrients in the basal media. For example, cells can be engineered to produce glutamine synthetase, which would reduce or eliminate the need for glutamine in the culture media formulation.

In certain embodiments, fish-specific versions of the growth factors mentioned herein may be used to for improved/enhanced function (relative to the typically used mammalian versions) when culturing adipogenic progenitor cells from fish or seafood.

In some cases, lactic acid may be extracted from the culture media (or first culture media or second culture media) and processed into polylactic acid. This beneficially repurposes a potential waste product.

As discussed elsewhere herein, the computational facilities of the system can include various artificial intelligence and machine learning algorithms to optimize production. Specifically, machine learning techniques that use a production variable as a target for optimization could work toward a more efficient culture media selection. The optimization process can be automated with appropriate robotics.

The present disclosure provides a host of end-use applications for the materials provided by the systems and methods described herein. These end-use applications can be applicable both to the desirable products produced by the systems and methods described herein (i.e., adipose tissue in many/most cases) and the undesirable or waste products produced by the systems and methods described herein (i.e., sedimented cells or non-adipose tissue in many/most cases), unless the context clearly dictates otherwise.

At a high level, the materials produced by the systems and methods described herein can generally be incorporated into edible products.

At a high level, the materials produced by the systems and methods described herein can generally be incorporated into consumer products not limited to cosmetics, skin creams, lotions, soaps, detergents, emulsifiers, lubricants, fuels, and/or animal feeds.

In one specific example, the materials produced by the systems and methods described herein can be included in cosmetics, such as make-up, skin creams, lotions, and the like.

In another specific example, the materials produced by the systems and methods described herein can be included in soaps, detergents, or other generally emulsifying compositions.

In another specific example, the materials produced by the systems and methods described herein can be included in industrial compositions, such as lubricants used in a variety of industries, liquids useful in extraction industries like the oil and gas industry, and the like.

In another specific example, the materials produced by the systems and methods described herein can be included in sustainability systems for environmental purposes, such as animal feeds, aquaculture compositions, and the like.

In another specific example, the materials produced by the systems and methods described herein can be included in end-use compositions that include some proportion of material that is produced from the systems and methods described herein and some proportion from naturally occurring sources. For instance, the cultured adipose tissue described herein can be combined with lean meat from a natural source to make ground meat having an artificially enhanced fat content.

In some cases, the materials produced can have components extracted and that can be the end product. For instance, the cultured adipose tissue can be rendered to remove fatty acids and those fatty acids can be the end product.

A system for executing the methods described herein is disclosed. The system includes one or more raw material sources. The raw material sources can feed raw materials into a first bioreactor. The first bioreactor can provide a first output to optionally proceed to a second bioreactor and can provide waste via a waste output to be collected for reuse and recycling. In some cases, the second bioreactor is not present and the first output is moved on to other portions of the system for further processing. The system can include any number of bioreactors. Those bioreactors can be arranged to operate in series, in parallel, or in any combination of the two. The system includes various extraction, filtration, and monitoring systems, as described elsewhere herein.

The raw material sources can be generally conventional material storage and supply technologies as would be appropriate for cell agriculture applications. One important requirement of the raw material sources is that the materials need to be sterile when they are eventually introduced into the system for executing the methods described above. Another optional feature of the raw material sources is temperature control. Particularly for the cellular material, the temperature control is important. Similarly, the conduits and other means of transport for the various material flows also require sterility and optionally include temperature controls.

The raw material sources can have quality control facilities. The raw material sources can have cellular testing facilities (e.g., PCR, morphology assessment, genetic testing, etc.). The raw material sources can have appropriate facilities to confirm sterility, such as sampling and testing facilities as would be understood by those having ordinary skill in the art.

The raw material sources can be adapted to sterilize the raw materials, using techniques known in the art.

At least one portion of the raw material sources is adapted to provide a supply of cells for use in methods described herein.

The raw material sources can take any physical form that meets the requirements of the applications described, including but not limited to, storage tanks, hoppers, reservoirs, silos, and other means of storing raw materials on-site for use in the methods described herein.

Receiving raw materials into the raw materials sources can be handled in generally conventional ways, with the understanding that the ordering and delivery of cells is an emerging field.

Without wishing to be bound by any particular theory, it is believed that the technology for preparing tailored cell lines such as the ones described elsewhere herein may be more accessible to the commercial market, so the system is adapted to receive commercial-scale delivery of cells as raw material for the methods described herein. As mentioned above, sterility is of paramount importance, as would be understood by a skilled artisan. With commercially provided cells, the supplier would provide information regarding the quality control of the materials, such as genetic information, instructions, etc.

In some cases, the raw material sources are fed by an on-site supply of raw materials.

In some cases, the raw material sources are supplied with locally sourced cells. In these cases, the system can include the necessary processing facilities to isolate cells of interest from livestock or other local sources of cells.

In some cases, the raw material sources for cells include cells that are embedded in a matrix, such as the natural tissue matrix from which they originated. In some cases, the cells used herein can be provided to the system in the form of agricultural silage mixtures.

The cells themselves may be extracted from agricultural waste products.

In some cases, the raw material sources are supplied or refilled via pipelines or other conduits for materials. In some cases, the raw material sources are themselves pipelines or other conduits for materials.

In some cases, the system include an entire sub-facility dedicated to producing cells for use in the methods described herein. This sub-facility can include all of the various processing systems that are required for processing agricultural and livestock products to isolate cells for use as described herein. In some cases, the systems may be paired with conventional farming and food distributions systems, such as butcheries or meat packing facilities. It should be appreciated that the initial stages of the growth of cellular agriculture are unlikely to supplant traditional meat sources, so the systems described herein may benefit from being integrated into a conventional food supply chain. In these cases, products that are typically viewed as waste could provide useful cells for use in the methods described herein.

In a general sense, the system can be divided into raw material production facilities, where cells and other reagents are produced, and end product production facilities, where the desired adipose tissue is produced.

The raw material sources can include sources for the various reagents used in the methods, including the culture media. As with the cellular material, the reagents used in the methods can also be locally sourced.

At least one portion of the raw material sources is adapted to provide culture media for use in the methods described herein. These raw material sources can include powdered storage media that is combined with purified water to make culture media or they can include the completed culture media itself.

The system is adapted to receive commercial-scale quantities of material, so the system includes receiving docks and pipe infrastructure to receive trailer trucks and tanker trucks of raw materials. Similarly, rail, air, and sea transport features are contemplated. As outlined above, pipelines are also a viable option for receiving raw material.

The raw materials can be recycled materials, including recycled agricultural materials and other recycled materials that can be useful in the processes described herein. In some cases, the raw materials are recycled from the system or method described herein.

The raw materials are delivered from the raw material sources into the first bioreactor. The means for delivery are conventional, with sterility maintained.

Material can be transferred between bioreactors using conventional delivery means, with sterility maintained.

Waste streams can be moved in conventional ways. However, the systems and methods described herein make improved use of waste. For example, most of the waste streams in this system and method can be recycled within the system.

Cellular material is transported throughout the system in a seed train, where cells move to progressively larger bioreactors. In this way, the seeding concentrations can be controlled and sterility may be maintained. The culture media in these bioreactors can be supplied in conventional ways.

Cellular material can be isolated from the culture media in ways understood to those having ordinary skill in the art, including centrifugation and filtration. One specific example of a device for separating cells from culture media is a counter flow centrifuge.

In some cases, the cells can be isolated from culture media by using micro-carrier, such as those made out of pectin.

The cells can be harvested using techniques made popular in different industries, such as the pharmaceutical industry and brewing industry, where cellular processes are used widely.

As discussed elsewhere herein, the cells can be harvested as floating cells. In these cases, certain specific harvesting devices can be used, which may not be applicable in other environments. For example. a skimmer could be used to skim the surface of a bioreactor to remove surface cells. Alternatively, a screen or mesh could be located beneath the surface of the culture media and then raised out of the culture media to harvest the floating cells while the culture media is drained and left behind.

As discussed elsewhere herein, the cells can be harvested as sedimented cells. In these cases, certain specific harvesting devices can be used, which may not be applicable in other environments. For example, suction filtration of a bottom portion of the bioreactor may be one suitable way to harvest sedimented cells.

In some cases, adhered cells can be harvested from a substrate using conventional methods. It should be appreciated that harvesting the adhered cells are not specifically the focus of most of this disclosure, though many of the features described herein are applicable to adhered cells. For the avoidance of doubt, the features of the present disclosure are also contemplated for use with harvested adhered cells.

Applicant has developed a technique for harvesting cells from the surface, which is different from existing techniques. Conventionally, when a layer of fat (cells or otherwise) accumulates on an aqueous surface, the layer is pipetted off the top by placing the tip of the pipette into the fat layer and introducing suction. However, this conventional process causes problems with certain samples and the fat layer cannot be isolated. To address this problem, the inventors surprisingly discovered that removing fat cells by maintaining suction on a pipette as the tip is drawn close to the fat layer, contacting the fat layer with the tip of the pipette, then lifting the pipette away from the surface, thereby removing a small section of the layer. This process is repeated for different locations on the layer, until most or all of the layer has been removed. This process can be manual or automated. If automated, a robotic arm with sensors can bring the pipette tip to the surface with adequate control to selectively remove the fat layer.

In some cases, the culture media within a given bioreactor is replaced with every usage. In some cases, the culture media is retained for some length of time before being replaced. In some cases, the culture media is slowly regenerated over time by supplementing the portions of the media that are removed in the process.

The system can include culture media filters, which filter culture media for reintroduction into the bioreactors.

The system can include an analytical device, such as optical spectrophotometer, a gas chromatography system, a mass spectrometry system, other analytical devices known in the art to useful for assessing quality control of liquid compositions such as culture media, or a combination thereof. This analytical device can be used to assess new or used culture media to determine if it is appropriate for further use.

In some cases, the analytical device is used to search for contaminants, such as undesirable bacterial growth or harsh chemical solvents. If contamination is identified, then the culture media can be routed to waste or to a refining facility for removal of the contaminants.

In some cases, the analytical device is used to confirm that certain desirable components are present. For example, the analytical device can confirm the presence or amount of one of the growth factors discussed above. If the analytical device identifies one or more desirable components are missing from a new or used culture media, then the culture media can be supplemented with the missing desirable components.

In some cases, the analytical device inspects for both contaminants and desirable components and directs the system to make the necessary remedial corrections, if needed.

In an aspect, the facility is equipped with the necessary scanning and tracking devices that would be required to keep track of the sourcing of the various components that go into the methods described herein. Without wishing to be bound by any particular theory, one of the powerful advantages that may be provided with cellular agriculture is the reduction in transportation costs that are required to transport meat from places where the animals are harvests to the consumer. In the case of the inventive system, the system can be located nearer to the consumer, so the bulk of the shipping cost and environmental impact is related to raw material shipping.

The system includes the necessary computing facilities to generate and/or modify blockchains in ways that are understood by those in the art to be useful for tracking the authenticity or sourcing of food products. Again, without wishing to be bound by any particular theory, it is believed that there may be advantage to providing consumers with evidence of how little shipping was required to produce a given food product. By using blockchain sourcing authentication techniques (or other methods known in the art to provide similar capabilities), the culture adipose tissue produced by the methods described herein can have corresponding entries into a blockchain regarding the cultured adipose tissue's provenance.

In addition to the digital provenance capability, the system and method described herein has the full capability to acquire and record any data that is necessary for complying with food regulatory authorities. Logs of processing parameters, test results, and other information that is relevant to regulators can be created and saved. The logs can be made manually or automatically.

In some cases, the digital records can be utilized to improve the system and method. In some cases, the improvements can be based on measured properties of the products, but in other cases, the improvements can be based on user feedback. There is inherently a lag between the facility producing a product and consumers enjoying it, so if there is a wave of product that has a particularly positive or negative customer response, then it would be possible to look up specific information from the run that led to that customer response for the purpose of intentionally repeating or not repeating it.

The data can include both information about the cell source material and the other formulation information from the process. If a certain cell line, process, and set of formulations produces a superior product, then the formula can be retrieved for reproduction.

Data can be collected from multiple facilities, thereby providing a global data set, from which broader conclusions can be drawn. For example, if all plants around the world show an inefficiency in the use of a given reagent, then this flags the problem as likely being related to the portions of the system or method that are deployed in all facilities.

In an aspect, the present disclosure provides a food product prepared directly from the culture adipose tissue that is made by the method described herein. This product stands in contrast to the other food products described herein, where the cultured adipose tissue is integrated with other components to make a food product that includes the cultured adipose tissue. The disclosed food product is a shaped and cooked piece of the cultured adipose tissue described herein. Without wishing to be bound by any particular theory, it is believed that the cultured adipose tissue disclosed herein can be treated as a food product with similar properties to certain animal products. In certain cases, slices of the cultured adipose tissue can be fried to form cultured adipose chips, which may resemble pork rinds in certain embodiments. In other cases, slices of the culture adipose tissue can be seared to form a seared fatty tissue morsel. In some cases, the cultured adipose tissue is cooked or fried. In some cases, the cultured adipose tissue is shaped to a particular three-dimensional configuration as desired.

It should be appreciated that the food products described herein, both those that integrate the culture adipose tissue and those that are composed entirely of cultured adipose tissue, can be adapted in the ways described above with respect to altering the nutritional or flavor contents, either by way of genetic manipulation or process manipulation. In this fashion, a previously unhealthy product can be adapted to provide a healthier alternative. For example, a cultured adipose tissue chip that is flavored to taste like pork rinds could be adapted to have a different fatty acid blend, thereby producing fewer negative health consequences to a consumer than a comparison conventional pork rind. Without wishing to be bound by any particular theory, a skilled artisan will recognize that comparisons regarding the health consequences of food may require larger studies and statistical averages rather than direct measurements of individual cases.

### EXAMPLES

### Example 1

### Timeline of 3T3-L1 Adipogenic Differentiation

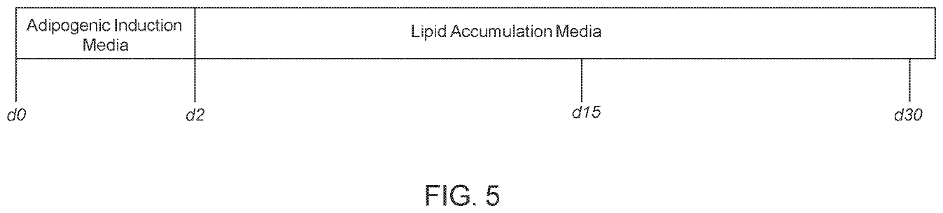

FIG. 5 shows a timeline for differentiation of 3T3-L1 adipogenic cells. Days 0 (d0), 2 (d2), 15 (d15), and 30 (d30) are indicated on the timeline. Confluent pre-adipocytes were grown in adipogenic induction media for the first two days and then switched to lipid accumulation media until harvest for cultured fat tissue formation on day 15 (see Example 2). Additional samples were grown in lipid accumulation media for 30 days to analyze lipid accumulation over longer-term culture.

Lipidomics and immunostaining were carried out on day 13. Additional samples were grown in lipid accumulation media for 1 month to analyze lipid accumulation over longer-term culture.

### Example 2

### Harvest of Lipid-Laden Adipocytes and Formation of 3D Cultured Fat Constructs

Following lipid accumulation, lipid-filled adipocytes were detached using a cell scraper. The adipocytes were then drained of non-cell liquid using a 0.22 micrometer vacuum filter. After detaching and draining, the in vitro adipocytes were then combined with transglutaminase or alginate and formed into discrete macroscale tissues in a 3D printed mold. Finally, 3D cultured fat constructs were mechanically tested for compressive strength, fluorescently stained for lipid and analyzed for volatile compounds.

### Example 3

### Methods for Generating 3D Cultured Fat Using Alginate or Transglutaminase

Alginate aggregation. Slow-gelling alginate solution was prepared by adding calcium carbonate and glucono delta-lactone (GDL) powders to a 1.6% or a 3.2% alginate solution before combining with the harvested and drained in vitro adipose tissue at a 1:1 volumetric ratio in a 3D printed mold.

Transglutaminase aggregation. Cultured fat was produced by mixing a 15% solution of transglutaminase with drained adipose tissue at a 2:8 volumetric ratio in a 3D printed mold.

### Example 4

### Generating Macroscale Cultured Fat Via the Aggregation of In Vitro Grown Adipocytes

Conventional animal agriculture for the production of meat (muscle and fat tissue) is linked with environmental degradation, zoonotic disease emergence, antimicrobial resistance, as well as animal welfare concerns. Methods to produce cultured (in vitro) fat tissue with reduced environmental impact and reduced negative impact on animals are thus important for providing the world with alternative approaches to producing these animal products. Here we have developed a method of producing bulk cultured adipose.

Native (in vivo) adipose tissue is largely a dense packing (aggregation) of lipid-filled adipocytes, held together by a sparse extracellular matrix (ECM) network. This is opposed to muscle tissue, which is comprised of aligned fibers in a multi-hierarchical structure. Since native adipose is an aggregation of adipocyte globules with less structural features than in muscle it is possible to recapitulate it on an organoleptic basis by aggregating separately grown adipocytes or adipocyte clusters. Growing adipocytes separately, or in small clusters, is desirable because it is currently infeasible to directly grow large tissues on the macroscale (millimeter scale and up) using contemporary tissue engineering techniques. By growing individual or small clusters of cells we are able to produce-at-scale a general mass of adipose cells, followed by the formation of actual adipose tissue through various methods of binding and aggregating the cells into a solid 3D construct. Methods of binding and aggregating the fat cells include enzymatic cross-linking with transglutaminase, as well as embedding in hydrogels such as alginate (a material which is already used as a fat replacer in the food industry).

An adipose aggregation approach circumvents the mass transport issue of macroscale 3D culture/tissue engineering by deliberately culturing individual adipocytes or small adipocyte clusters via 2D culture or various scalable bioreactors. Only after the separated adipocytes have grown and accumulated sufficient lipid does cell culture end, followed by the manual aggregation of the cultured cells into denser 3D tissues without the need for vascularization and perfusion. This is uniquely possible with cultured fat because for food purposes, for example, once the tissue is formed and harvested the cultured cells do not need to stay alive. This process is analogous to meat production in conventional animal agriculture where muscle and fat cells gradually cease to be viable after slaughter. For conventional tissue engineering (e.g., medical applications etc.) cells in 3D tissues are expected to remain viable to be used for implantation into the body, or for use and testing in an in vitro tissue model.

### Example 5

### Avoiding Lift-Off Issues Faced With Adherent Adipocytes

Applicant has also observed that a large subpopulation of the cultured adipocytes adhere strongly to tissue culture plates and do not float away, avoiding issues of lift-off of adherent adipocytes in vitro due to increasing buoyancy as the adipocytes become fatty. The 2D culture systems self-sort for adherent cell populations. However, techniques have been developed for dealing with the non-adherent cell populations.

It is believed that analytical (e.g., optical, etc.) techniques that interrogate individual cells can provide meaningful distinctions between adipose cells that are ripe for processing/harvesting versus adipose cells that are not yet ready. For example, individual optical interrogation of cells revealing underlying lipid accumulation can be performed, followed by comparing that interrogation to a benchmark threshold for cells known to have a desired lipid accumulation (or other measurable property). Alternatively or in addition, populations of cells can have optical measurements taken (e.g., absorption, scattering measurements, etc.) or electrical measurements taken (e.g., impedance, capacitance, etc.), which can represent important meaningful properties of the population of cells. An assessment of such benchmark threshold or property/properties allows for the processing/harvesting decision to select an appropriate time for processing/harvesting cells (or cell). This decision can be made based on cell growth ((i.e., cell size and/or mass and/or density, number of cells or cell count).

In some instances, there is no need to examine adhered cells for harvesting decisions, because their adherence is a reflection of inadequate fat content. In other words, if the adherence is tuned to a given general adherence, then detachment can serve as the sign that the cell is ready for harvest. However, we found that monitoring of non-adhered cells performed better for uses herein.

It is believed that lipid droplet size is an important factor in determining when an adipose cell is ready for harvesting. This lipid droplet size is optically visible and can be interrogated by known optical methods. As such, monitoring of lipid droplet size within a cell or within a population of cells could be used for harvesting decisions.

We also propose monitoring the culture media to make harvesting decisions. Notably, ammonia and/or lactic acid concentrations could be used as the monitoring metric. The cell secretome can also be analyzed. Specifically, the level of adipokine or adiponectin surrounding the cells can be used as a measure of readiness for harvest.

We also propose assigning grades to cells based on density. For example, cells having a lower density can be assigned a higher grade. Cells that remain adhered to a substrate for a shorter length of time are assigned a higher quality grade. This grade can be used for harvesting decisions, as well.

In general, monitoring can be performed on non-adhered (or “floating”) cells. In the case where multiple different density gradients are deployed and/or where multiple different populations of floating cells are collected, these cells can be divided into quality grades based on one or more properties as discussed above. Floating cells can be delivered to a number of useful devices including chromatographic systems, microfluidics systems, cell sorting systems, cell pickers, and cell skimmers, to name a few examples.

In cases where the floating cells are directed to the microfluidics system, the microfluidics system can be tailored specifically for handling floating cells described herein. One example of tailoring the microfluidics system for floating cells is to adjust the density of the carrier fluid prior to entry into the microfluidics system, such that the cells are neutral buoyance and are no longer “floating” in the medium.

Some means for harvesting, aggregating, and/or collecting floating cells are appreciated in the art. However, Applicant proposes harvesting or collecting floating cells by skimming them from culture media. As adhered cells remain fixed to the surface of the bioreactor or culture substrate, floating cells prove capable of being skimmed from the culture media. Skimming can be performed by means known in the art. Applicant proposes one such method as described in Example 9. Other methods of harvesting are at least disclosed in Dryden et al., “Technical and Economic Considerations of Cell Culture Harvest and Clarification Technologies” (Biochem Eng J, Vol. 167, 107892) and elsewhere. Prior to molding the harvested cells in the next portion of the method, some post-processing may be required. For example, the harvested cells might be rinsed to remove/minimize/dilute undesired culture media components. Rinsing can be done by way of counter-flow centrifugation or conventional filtration. As another example, cells can be clarified by removing undesirable components, which are not removed by rinsing. This is a process that is regularly used in the pharmaceutical arts, but it is not conventionally used in cellular agriculture. In one specific example, clarification of cells to remove cell debris or excess nucleic acids is expressly contemplated. Other methods such as flocculation via pH or the use of polymers such as chitosan which can induce flocculation are contemplated. Other methods of collecting non-adhered cells may comprise collecting non-clustered floating cells, collecting only non-adhered cells, and/or not collecting adhered cells.

In cases where the floating cells are directed to the chromatography system, a specific stationary phase and mobile phase may be selected for a desired result. For instance, if size selection is an important criterion, then a stationary phase that has porosity that provides differentiated retention properties based on size could be useful. As another example, if surface chemistry of the adipose cells is an important criterion, then a stationary and/or mobile phase could be selected based on those desirable surface chemistries. In one specific example, certain cells may be more amenable to embedding into a hydrogel if they have a specific surface chemistry, so a chromatography system could be used to isolate cells that are more or less amenable to embedding into the hydrogel. Similarly, in another specific example, certain cells may be more amenable to direct cross-linking to other cells if they have a specific surface chemistry, so a chromatography system could be used to isolate cells that are more or less amenable to direct cross-linking with one another.

In cases where the floating cells are directed to a cell sorting system, the cell sorting system can be used to differentiate and categorize the cells based on measurable properties, as would be appreciated by a skilled artisan. Applicant does not intend to provide specific inventive contribution with respect to the cell sorting itself, but rather integrates and utilizes the understood concept of cell sorting into inventive concepts that form a part of the broader disclosure described herein.

In some cases, the cells are sorted through multiple different centrifuges. In that case, the cells that make it through multiple centrifuges will have different properties from those that do not and those different categories of cells can be divided into grades, in a similar fashion as USDA grading. The cells could be graded as superior or inferior relative to one another.

The systems and methods of this disclosure provide approaches to collecting and utilizing these floating cells, which have traditionally been discarded as waste. By making productive use of these previously wasted products, much higher efficiency is achieved by virtue of not wasting this large population of cells.

The same techniques described above may be inversely applicable to sedimented cells. For example, rather than monitoring a surface interface, the bottom of a reaction tank could be monitored for sedimented cells. Sedimented cells may be preferred for certain applications and those sedimented cells can be collected and further processed for those applications. However, given the focus of adipose tissue, which is generally buoyant in aqueous environments such as growth cultures, the sedimented cells are typically waste products and can be processed as such.

Numerous other metrics could be utilized in this method to monitor readiness of cells for harvest.

### Example 6

### Utilizing Cultured Sedimented Cells

As described in Example 5, the techniques described herein may result in waste products—which may include sedimented cells—which may possess utility elsewhere.

Sedimented cells can be isolated and further processed as biomass or recycled for raw materials and reintroduced into the system and/or method at various points in the system/process. For example, sedimented cells can be harvested and have components for culture media extracted from them. In certain cases sedimented cells are harvested separately from non-adhered cells. Other uses may include filler material in animal feedstock, starting material for a further culture, and/or a single cell protein for growth of bacteria or fungus.

One disadvantage of sedimented cells is that they are typically very high in nucleic acid content. In some cases, the sedimented cells can have nucleic acid content reduced before further use. However, the cells can still be useful without reducing the nucleic acid content, for example, as livestock feed. In some cases, the sedimented cells, with or without reducing the nucleic acid content, can be mixed into one or more of the food products described herein for the purpose of adding more of a “meat” flavor.

### Example 7

### Advantages in Culturing Adipocytes

The techniques described herein can provide advantageous ability to administer growth factors to the cells, in the interest of providing them with more optimal receipt and uptake of the growth factors. Growth factors and other additives can be incorporated into the hydrogel or binder in which harvested adipose cells 12 are aggregated. These additives can include flavorants, colorants, texturizers, vitamins, minerals, amino acids, proteins/peptides, and fatty acids and can be added to the hydrogel or binder in amounts that are tuned to the desired outcome. Growth factors that are suitable for use as additives can include fibroblast growth factor 2, transforming growth factor beta 3, and insulin-like growth factor. It is believed that cultured cells are generally deficient in oleic acid, linoleic acid, and/or arachidonic acid. Thus, these can all be additives, thereby obviating the deficiency.

One particularly useful additive for the present disclosure is a fatty acid. It is believed that cell lines that are generally lacking in the production of a desirable fatty acid can be supplemented in the development process to include more of the desirable fatty acid and that in vitro fat cells have historically been lacking in certain fatty acids. The most abundantly missing fatty acid is linoleic acid (18:2), as mammals take this from the diet rather than synthesizing it themselves. There can also be a lack of arachidonic acid (20:4), as it is made from 18:2. The lack of 20:4 can be both good and bad, as it's been reported to be important for flavor but also a precursor molecule to pro-inflammatory compounds (eicosanoids). In vitro mammalian cells are also typically lacking in omega 3 fatty acids as these are also dietary. These would be alpha-linolenic acid (18:3), eicosapentaenoic acid (EPA, 20:5), and docosahexaenoic acid (DHA, 22:6). Omega 3s are generally less abundant in terrestrial (land) animals so a lack of them is not that detrimental. However, they can make up a significant proportion of fish lipids, so it may be important to have for fish adipocytes. In a general sense, when multiple different fatty acids are supplemented, the supplementing concentrations can be chosen to provide desirable end concentrations.

In one example, soybean oil can be added. Soybean oil has been shown to increase lipid drop size in in vitro adipose tissue. By adding soybean oil, a desired droplet size can be achieved more quickly, thereby enhancing efficiency of the overall process.

Certain additives may be present in the form of an uptake-enhancing additive for the explicit purpose of enhancing uptake of other additives. In a mouse model, the uptake of triglycerides was enhanced by administering an agent that induced the release of a relevant lipase, which allowed the breakdown of the triglycerides for entry into the cells. Co-administered agents may be required to be administered with a given additive in the interest of improving uptake of the additive. The uptake-enhancing additive can be an enzyme that modifies a different additive to make that different additive more suitable for uptake (e.g., a lipase with large lipids). The uptake-enhancing additive can be a permeability-enhancing agent, which enhances the permeability of the cell membrane of the adipose cells.

One specific avenue for introducing growth factors and/or agents and/or additive into the inventive compositions is by way of adding the growth factors and/or agents and/or additives into the first culture media or the second culture media at the time that those culture media are made and/or when those culture media are introduced into their respective tanks.

The culture media can be tailored for proliferation and differentiation, as well for other steps of the process. In addition, the various steps can use more than one culture media. For example, one culture media could be used for a first part of a proliferation process and the cells can be transferred to a different culture media for a later part of the proliferation process.

The culture media is typically comprised of a basal medium containing basic nutrients (sugars/carbohydrates, lipids/fatty acids, amino acids, vitamins, minerals, salts, water) combined with a growth factor or complex component containing proteins, peptides, hormones, or other bioactive compounds responsible for directing cell behavior. (e.g., to promote proliferation/cell survival, or to promote adipogenesis, or to suppress other cell pathways such as osteogenesis). Basal media will likely be best optimized for the specific adipogenic progenitor cell that is used but may be similar to or based off of existing formulations such as Dulbecco's modified eagle medium (DMEM), Ham's F12, Ham's MCDB 131, Iscove's modified Dulbecco's medium (IMDM).

Growth factors for adipogenic progenitor proliferation media formulations may include non-exhaustively: serum albumins, basic fibroblast growth factor (bFGF), epidermal growth factor (EGF), platelet derived growth factor (PDGF), fetuin, bone morphogenic proteins (BMPs, e.g., BMP4), Wnt proteins, leukemia inhibitor factor (LIF), hydrocortisone, testosterone, progesterone, estrogen, hypoxia inducible factors (HIFs), stem cell growth factor-β (SCGF-β), tumor necrosis factor alpha (TNFα), interleukin-1 beta (IL-1β) and other interleukins (ILs), transforming growth factor beta (TGFβ), insulin-like growth factor 1 (IGF-1), insulin, transferrin, myoglobin, vitronectin, truncated vitronectin peptides, laminin, laminin peptides (e.g., 511-E8), fibronectin, collagen, gelatin, trypsin inhibitors.

Growth factors specifically for adipogenic progenitor cell differentiation include non-exhaustively: Phospholipids, cholesterols (e.g., very low density lipoprotein, VLDL), fatty acids (e.g., oleic acid, linoleic acid), triacylglycerides (e.g., soybean oil, olive oil, rapeseed oil, fish oil), cyclodextrins (to solubilize non-polar ingredients), PPARγ agonists (e.g., rosiglitazone, pioglitazone, pristanic acid, phytanic acid, lutein, beta-carotene, all-trans retinoic acid, 9-cis retinoic acid), ascorbic acid, isobutylmethylxanthine (IBMX), dexamethasone, hydrocortisone, insulin, insulin-like growth factor 1 (IGF-1). In some cases, components that promote differentiation may overlap with ingredients in the basal medium formulation.

### Example 8

### A System for Culturing Adipocytes

A system for culturing adipocytes is disclosed herein. The system includes one or more raw material sources feeding into a first bioreactor. The first bioreactor can provide a first output to optionally proceed to a second bioreactor and can provide waste via a waste output to be collected for reuse and recycling. A second bioreactor may not be present and the first output is moved on to other portions of the system for further processing. The system can include any number of bioreactors. Those bioreactors can be arranged to operate in series, in parallel, or in any combination of the two. The system includes various extraction, filtration, and monitoring systems. The raw material sources can be generally conventional material storage and supply technologies as would be appropriate for cell agriculture applications. One important requirement of the raw material sources is that the materials need to be sterile when they are eventually introduced into the system for executing the methods described above. Another optional feature of the raw material sources is temperature control. Particularly for the cellular material, the temperature control is important. Similarly, the conduits and other means of transport for the various material flows also require sterility and optionally include temperature controls.

The raw material sources can have quality control facilities, cellular testing facilities (e.g., PCR, morphology assessment, genetic testing, etc.), and/or appropriate facilities to confirm sterility, such as sampling and testing facilities. Importantly, the raw material sources can be adapted to sterilize the raw materials. At least one portion of the raw material sources is adapted to provide a supply of cells for use in the methods described herein. The raw material sources can take any physical form that meets the requirements of the applications described, including but not limited to, storage tanks, hoppers, reservoirs, silos, and other means of storing raw materials on-site for use in the methods described herein. Receiving raw materials into the raw materials sources can be handled in generally conventional ways, with the understanding that the ordering and delivery of cells is an emerging field. The system is adapted to receive commercial-scale delivery of cells as raw material for the methods described herein. As mentioned above, sterility is of paramount importance. With commercially provided cells, the supplier would provide information regarding the quality control of the materials, such as genetic information, instructions, etc. The raw material sources may be fed by an on-site supply of raw materials and/or with locally sourced cells. The system can include the necessary processing facilities to isolate cells of interest from livestock or other local sources of cells. The raw material sources for cells may include cells that are embedded in a matrix, such as the natural tissue matrix from which they originated and may be provided to the system in the form of agricultural silage mixtures. The cells themselves may be extracted from agricultural waste products.

The system may include an entire sub-facility dedicated to producing cells for use in the methods described herein. This sub-facility can include all of the various processing systems that are required for processing agricultural and livestock products to isolate cells The systems may be paired with conventional farming and food distributions systems, such as butcheries or meat packing facilities. It should be appreciated that the initial stages of the growth of cellular agriculture are unlikely to supplant traditional meat sources, so the systems described herein may benefit from being integrated into a conventional food supply chain. In these cases, products that are typically viewed as waste could provide useful cells for use in the methods described herein.

In a general sense, the system can be divided into raw material production facilities, where cells and other reagents are produced, and end product production facilities, where the desired adipose tissue is produced. At least one portion of the raw material sources is adapted to provide culture media for use in the methods described herein. These raw material sources can include powdered storage media that is combined with purified water to make culture media or they can include the completed culture media itself.

The system is adapted to receive commercial-scale quantities of material, so the system includes receiving docks and pipe infrastructure to receive trailer trucks and tanker trucks of raw materials. Similarly, rail, air, and sea transport features are contemplated. As outlined above, pipelines are also a viable option for receiving raw material. The raw materials can be recycled materials, including recycled agricultural materials and other recycled materials that can be useful in the processes described herein. In some cases, the raw materials are recycled from the system or method described herein.

The raw materials are delivered from the raw material sources into the first bioreactor. The means for delivery into the first bioreactor and for the transfer between bioreactors are conventional, with sterility maintained.

Waste streams can be moved in conventional ways. However, the systems and methods described herein make improved use of waste. For example, most of the waste streams in this system and method can be recycled within the system.

Cellular material is transported throughout the system in a seed train, where cells move to progressively larger bioreactors. In this way, the seeding concentrations can be controlled and sterility may be maintained. The culture media in these bioreactors can be supplied in conventional ways.

Cellular material can be isolated from the culture media in ways understood to those having ordinary skill in the art, including centrifugation and filtration. One specific example of a device for separating cells from culture media is a counter flow centrifuge. The cells can be isolated from culture media by using micro-carrier, such as those made out of pectin. The cells can be harvested using techniques made popular in different industries, such as the pharmaceutical industry and brewing industry, where cellular processes are used widely. As discussed in Example 7, the cells can be harvested as floating cells. In these cases, certain specific harvesting devices can be used, which may not be applicable in other environments. For example. a skimmer could be used to skim the surface of a bioreactor to remove surface cells. Alternatively, a screen or mesh could be located beneath the surface of the culture media and then raised out of the culture media to harvest the floating cells while the culture media is drained and left behind. As discussed in Example 7 and Example 8, the cells can be harvested as sedimented cells. In these cases, certain specific harvesting devices can be used, which may not be applicable in other environments. For example, suction filtration of a bottom portion of the bioreactor may be one suitable way to harvest sedimented cells.

The culture media within a given bioreactor is replaced with every usage. In some cases, the culture media is retained for some length of time before being replaced. In some cases, the culture media is slowly regenerated over time by supplementing the portions of the media that are removed in the process. The system can include culture media filters, which filter culture media for reintroduction into the bioreactors. The system can include an analytical device, such as optical spectrophotometer, a gas chromatography system, a mass spectrometry system, other analytical devices known in the art to useful for assessing quality control of liquid compositions such as culture media, or a combination thereof. This analytical device can be used to assess new or used culture media to determine if it is appropriate for further use. The analytical device may be used to search for contaminants, such as undesirable bacterial growth or harsh chemical solvents. If contamination is identified, then the culture media can be routed to waste or to a refining facility for removal of the contaminants. The analytical device may be used to confirm that certain desirable components are present. For example, the analytical device can confirm the presence or amount of one of the growth factors discussed above. If the analytical device identifies one or more desirable components are missing from a new or used culture media, then the culture media can be supplemented with the missing desirable components. In some cases, the analytical device inspects for both contaminants and desirable components and directs the system to make the necessary remedial corrections, if needed.

The facility may be equipped with the necessary scanning and tracking devices that would be required to keep track of the sourcing of the various components that go into the methods described herein. Without wishing to be bound by any particular theory, one of the powerful advantages that may be provided with cellular agriculture is the reduction in transportation costs that are required to transport meat from places where the animals are harvests to the consumer. In the case of the inventive system, the system can be located nearer to the consumer, so the bulk of the shipping cost and environmental impact is related to raw material shipping.

The system includes the necessary computing facilities to generate and/or modify blockchains in ways that are understood by those in the art to be useful for tracking the authenticity or sourcing of food products. Again, without wishing to be bound by any particular theory, it is believed that there may be advantage to providing consumers with evidence of how little shipping was required to produce a given food product. By using blockchain sourcing authentication techniques (or other methods known in the art to provide similar capabilities), the culture adipose tissue produced by the methods described herein can have corresponding entries into a blockchain regarding the cultured adipose tissue's provenance.

In addition to the digital provenance capability, the system and method described herein has the full capability to acquire and record any data that is necessary for complying with food regulatory authorities. Logs of processing parameters, test results, and other information that is relevant to regulators can be created and saved. The logs can be made manually or automatically. The digital records can be utilized to improve the system and method. In some cases, the improvements can be based on measured properties of the products, but in other cases, the improvements can be based on user feedback. There is inherently a lag between the facility producing a product and consumers enjoying it, so if there is a wave of product that has a particularly positive or negative customer response, then it would be possible to look up specific information from the run that led to that customer response for the purpose of intentionally repeating or not repeating it. The data can include both information about the cell source material and the other formulation information from the process. If a certain cell line, process, and set of formulations produces a superior product, then the formula can be retrieved for reproduction. Data can be collected from multiple facilities, thereby providing a global data set, from which broader conclusions can be drawn. For example, if all plants around the world show an inefficiency in the use of a given reagent, then this flags the problem as likely being related to the portions of the system or method that are deployed in all facilities.

### Example 9

### A Technique for Harvesting Cells From the Surface

Applicant has developed a technique for harvesting cells from the surface, which is different from existing techniques. Conventionally, when a layer of fat (cells or otherwise) accumulates on an aqueous surface, the layer is pipetted off the top by placing the tip of the pipette into the fat layer and introducing suction. However, this conventional process causes problems with certain samples and the fat layer cannot be isolated. To address this problem, the inventors surprisingly discovered that removing fat cells by maintaining suction on a pipette as the tip is drawn close to the fat layer, contacting the fat layer with the tip of the pipette, then lifting the pipette away from the surface, thereby removing a small section of the layer. This process is repeated for different locations on the layer, until most or all of the layer has been removed. This process can be manual or automated. If automated, a robotic arm with sensors can bring the pipette tip to the surface with adequate control to selectively remove the fat layer.

### Example 10

### Aggregation of Adipose Cells by Flocculation

Applicant proposes the aggregation of non-adhered adipose cells by flocculation. Flocculation often refers to the aggregation of unstable and small particles through surface charge neutralization, electrostatic patching and/or bridging after addition of flocculants (or agents that make fine and sub-fine solids or colloids suspended in the solution form large loose flocs through bridging thus achieving solid-liquid separation). Flocculation may be performed by one or more of several techniques, including auto-flocculation, bio-flocculation, chemical flocculation, particle-based flocculation, and electrochemical flocculation. Various flocculation techniques have been reviewed by Matter, et al (2019, Applied Sciences). This is a method with high potential as it could be used to harvest cells from the bioreactor at low cost.

Auto-flocculation may be performed at acidic pH (for example, a pH of 4.0). Auto-flocculation may be performed at alkaline pH (for example, a pH of 10.4, 11.0, 1.5, 11.6, 12.0, or 12.5). Auto-flocculation may be performed over several days (for example, 16 days).

Chemical flocculation may be performed with inorganic flocculants such as aluminum sulfate, calcium oxide, iron(III) chloride, and magnesium hydroxide. Chemical flocculation may be performed with organic flocculants such as inulin, starches, chitosan, tannins, alginates, and lysines. Food-safe flocculants (such as chitosan and other long polymers) may be preferred.

Particle-based flocculation may be performed using aminoclay-based nanoparticles such as 3-aminopropyltriethoxysilane (APTES) conjugated to magnesium and aminoclays conjugated to aluminum, titanium oxide, and humic acid. Particle-based flocculation may be performed using magnetic particles including ferrous oxide nanoparticles and composites.

Adipose cells aggregated through means discussed in the present example may be further processed or used in the various methods, food products, systems (etc.) described in the present application.

### Example 11

### Food Product Prepared by Adipose Tissue Made by the Methods Herein

A food product may be prepared directly from the culture adipose tissue that is made by the method described herein. This product stands in contrast to the other food products described herein, where the cultured adipose tissue is integrated with other components to make a food product that includes the cultured adipose tissue. The disclosed food product is a shaped and cooked piece of the cultured adipose tissue described herein. It is believed that the cultured adipose tissue disclosed herein can be treated as a food product with similar properties to certain animal products. In certain cases, slices of the cultured adipose tissue can be fried to form cultured adipose chips, which may resemble pork rinds in certain embodiments. In other cases, slices of the culture adipose tissue can be seared to form a seared fatty tissue morsel.

### Additional Examples

1. A method for producing cultured adipose tissue, comprising:
growing adipogenic precursor cells in a first culture media;
differentiating the adipogenic precursor cells to adipose cells in a second culture media;
harvesting non-adhered adipose cells; and
aggregating the harvested non-adhered adipose cells to provide the cultured adipose tissue.

2. The method of case 1, wherein growing the adipogenic precursor cells in the first culture media comprises seeding the adipogenic precursor cells into a bioreactor containing the first culture media and allowing the adipogenic precursor cells to proliferate in the bioreactor.

3. The method of case 2, wherein differentiating the adipogenic precursor cells to adipose cells comprises changing the first culture media to the second culture media in the bioreactor.

4. A method for producing cultured adipose tissue, comprising:

growing adipogenic precursor cells in a culture media; differentiating the adipogenic precursor cells to adipose cells in the culture media; harvesting non-adhered adipose cells; and aggregating the harvested non-adhered adipose cells to provide the cultured adipose tissue.

5. The method of cases 1 or 4, wherein the method is carried out in a bioreactor.

6. The method of cases 2, 3 or 5, wherein the bioreactor is selected from the group consisting of a stirred suspension tank bioreactor, airlift bioreactor, bubble column bioreactor, fluidized bed bioreactor, packed bed bioreactor, vertical wheel bioreactor, and wave bag bioreactor.

7. The method of cases 2, 3 or 5, wherein the bioreactor is a rotating wall vessel bioreactor.

8. The method of cases 2, 3 or 5, wherein the bioreactor is a hollow fiber bioreactor.

9. A method for producing cultured adipose tissue, comprising:
growing adipogenic precursor cells on a two-dimensional (2D) substrate;
differentiating the adipogenic precursor cells to adipose cells on the 2D substrate;
harvesting non-adhered adipose cells; and
aggregating the harvested non-adhered adipose cells to provide the cultured adipose tissue.

10. The method of case 9, wherein growing the adipogenic precursor cells on the 2D substrate comprises seeding the adipogenic precursor cells onto the 2D substrate and allowing the adipogenic precursor cells to proliferate on the 2D substrate.

11. The method of cases 9 or 10, wherein the 2D substrate forms at least a portion of a conveyor belt.

12. The method of any of cases 9 to 11, wherein the method is performed continuously in an assembly line process.

13. A method for producing cultured adipose tissue, comprising:
culturing adipose cells from adipogenic precursor cells in culture media;
harvesting non-adhered adipose cells after a predetermined or desired amount of adipose cells are produced; and
aggregating the harvested non-adhered adipose cells to provide the cultured adipose tissue.

14. The method of any one of the preceding cases, wherein the adipogenic precursor cells are pluripotent stem cells.

15. The methods of any of cases 1 to 13, wherein the adipogenic precursor cells are selected from the group consisting of mesenchymal stem cells, dedifferentiated fat cells, fibroblasts, and fibroadipogenic progenitor cells.

16. The method of any one of the preceding cases, wherein aggregating the harvested non-adhered adipose cells comprises mixing the harvested non-adhered adipose cells with a hydrogel or binder in a 3D mold.

17. The method of case 16, wherein the hydrogel or binder is selected from the group consisting of alginate, cellulose, gelatin, starch, tara gum, agar, agarose, carrageenan, cornstarch, gellan gum, lecithin, maltodextrin, methylcellulose, carboxymethylcellulose, cellulose gum, hydroxypropyl cellulose, hydroxypropyl methylcellulose tragacanth, karaya, acacia, ghatti, beta-glucan, psyllium husk, inulin, chitosan, curdlan, dextran, potato starch, tapioca/arrowroot starch, hyaluronic acid, fibrin, carrageenan, guar gum, inulin, konjac, oat bran, pectin, locust bean gum, xanthan gum, soy protein, wheat protein, pea protein, chickpea protein, almond protein, rice protein, hemp protein, quinoa protein, sunflower protein, potato protein, algae protein, yeast protein, bacterial protein, corn protein, silk protein, cellulose derivatives, pullulan, hydrolysates of any of the aforementioned proteins, and combinations thereof.

18. The method of case 16 or 17, further comprising incorporating at least one growth factor or at least one additive into the hydrogel or binder.

19. The method of any one of the preceding cases, wherein aggregating the harvested non-adhered adipose cells comprises mixing the harvested non-adhered adipose cells with alginate.

20. The method of case 19, wherein mixing the harvested non-adhered adipose cells with alginate comprises:
adding calcium carbonate and glucono delta-lactone to a solution of the alginate; and
combining the harvested non-adhered adipose cells with the solution of the alginate in the 3D mold.

21. The method of any one of the preceding cases, further comprising cross-linking the harvested non-adhered adipose cells.

22. The method of case 21, wherein cross-linking the harvested non-adhered adipose cells comprises cross-linking the harvested non-adhered adipose cells using an enzyme selected from the group consisting of a transglutaminase, a tyrosinase, a peroxidase, a laccase, a sortase, a subtilisin, and lysyl oxidase.

23. The method of case 21, wherein cross-linking the harvested non-adhered adipose cells comprises enzymatically cross-linking the harvested non-adhered adipose cells using transglutaminase.

24. The method of case 23, wherein cross-linking the harvested non-adhered adipose cells with transglutaminase comprises mixing a solution of transglutaminase with the harvested non-adhered adipose cells at a predetermined volumetric ratio.

25. The method of case 21, wherein cross-linking the harvested non-adhered adipose cells comprises cross-linking the harvested non-adhered adipose cells using a cross-linker selected from the group consisting of polymers functionalized with aldehyde groups, genipin, and phenolic compounds.

26. The method of case 25, wherein the polymers functionalized with aldehyde groups are selected from a group consisting of periodate oxidized pectin, dextran, chitosan, Arabic gum, sucrose, raffinose, stachyose, cyclodextrin, and starch.

27. The method of case 25, wherein the phenolic compound is selected from the group consisting of caffeic acid, chlorogenic acid, caftaric acid, quercetin, and rutin.

28. The method of any one of the preceding cases, wherein aggregating the harvested non-adhered adipose cells comprises adding a protein during the aggregation.

29. The method of case 28, wherein the protein is selected from the group consisting of casein, gelatin, pea protein, soy protein, wheat protein, corn protein, chickpea protein, almond protein, rice protein, hemp protein, quinoa protein, sunflower protein, potato protein, algae protein, yeast protein, bacterial protein, mycoprotein, hydrolysates of any of the aforementioned proteins, and combinations thereof.

30. The method of any one of cases 1 to 29, wherein the harvesting and/or the aggregating steps comprise flocculation or coagulation of the non-adhered adipose cells.

31. The method of case 30, wherein flocculation comprises a flocculation technique selected from the group comprising auto-flocculation, bio-flocculation, chemical flocculation, particle-based flocculation, and electrochemical flocculation 32. The method of case 30, wherein flocculation is performed with a food-safe flocculant or coagulant.

33. The method of case 32, wherein the food-safe flocculant or coagulant is selected from the group consisting of chitosans, celluloses, lignins, inulins, starches, tannins, alginates, lysines, long polymers, and combinations thereof.

34. The method of any one of the preceding cases, further comprising draining the adipose cells to remove non-cell liquid after harvesting the adipose cells and prior to aggregating the harvest non-adhered adipose cells.

35. The method of any one of the preceding cases, wherein the cultured adipose tissue has a size on the macroscale.

36. The method of any one of the preceding cases, wherein the cultured adipose tissue has a defined 3D shape.

37. The method of any one of the preceding cases, further comprising supplementing the adipose cells with methylated branched fatty acids.

38. The method of any one of the preceding cases, wherein the adipose cells express omega 3 desaturases.

39. The method of any one of the preceding cases, further comprising monitoring the harvested non-adhered adipose cells for lipid accumulation prior to harvesting.

40. The method of case 39, wherein monitoring comprises optical or electrical monitoring.

41. The method of case 39, wherein monitoring comprises measuring lipid droplet size within a cell or within a population of cells.

42. The method of any one of the preceding cases, further comprising determining a concentration of ammonia and/or lactic acid in the culture media prior to harvesting the non-adhered adipose cells.

43. The method of any one of the preceding cases, further comprising extracting lactic acid from the culture media and processing the lactic acid into polylactic acid.

44. The method of any one of the preceding cases, further comprising determining the level of adipokine and/or adiponectin in the culture media prior to harvesting the non-adhered adipose cells.

45. The method of any one of the preceding cases, further comprising administering at least one growth factor or at least one additive to the adipogenic precursor cells.

46. The method of case 18 or 45, wherein the at least one growth factor is selected from the group comprising fibroblast growth factor 2, transforming growth factor beta 3, insulin-like growth factor.

47. The method of case 46, wherein the at least one growth factor is fish-specific.

48. The method of case 18 or 45, wherein the at least one additive is selected from the group comprising flavorants, colorants, texturizers, vitamins, minerals, amino acids, proteins/peptides, whole foods, oils, and fatty acids.

49. The method of case 46, wherein the at least one additive is a fatty acid selected from the group comprising linoleic acid, arachidonic acid, alpha-linolenic acid, eicosapentaenoic acid, and docosahexaenoic acid.

50. The method of case 16, wherein the at least one additive is selected from the group comprising soybean oil, coconut oil, palm oil, cocoa butter, shea butter, mango butter, olive oil, canola oil, sunflower oil, flaxseed oil, avocado oil, peanut oil, sesame oil, corn oil, vegetable oil, margarine, shortening, and vegetable ghee.

51. The method of case 50, wherein the at least one additive is hydrogenated at least partially.

52. The method of case 50 or 51, wherein the at least one additive is in oleogel, bigel, and/or emulgel form.

53. The method of case 50, wherein the at least one additive is a whole food selected from the group comprising tofu, tempeh, seitan, jackfruit, quinoa, chia seeds, bananas, oats, avocados, flax seeds, rice, tapioca/arrowroot, and potatoes.

54. The method of any one of the preceding cases, further comprising harvesting sedimented cells.

55. The method of case 54, further comprising processing the sedimented cells into biomass.

56. The method of any one of the preceding cases, further comprising reducing the nucleic acid content prior to harvesting the non-adhered adipose cells.

57. The method of any one of the preceding cases, wherein harvesting the non-adhered adipose cells comprises skimming non-adhered cells.

58. The method of case 6, wherein harvesting the non-adhered adipose cells consists of collecting non-clustered floating cells.

59. The method of any one of the preceding cases, wherein harvesting the non-adhered adipose cells consists of collecting only non-adhered adipose cells.

60. The method of any one of the preceding cases, wherein harvesting the non-adhered adipose cells does not collect adhered cells.

61. A cultured adipose tissue produced by the method of any one of the preceding cases.

62. A cultured adipose tissue comprising adipose cells embedded in a hydrogel or binder, wherein the cultured adipose tissue has a three-dimensional (3D) shape and a size on the macroscale.

63. The cultured adipose tissue of case 62, wherein the hydrogel or binder is selected from the group consisting of alginate, cellulose, gelatin, starch, tara gum, agar, agarose, carrageenan, cornstarch, gellan gum, lecithin, maltodextrin, methylcellulose, carboxymethylcellulose, cellulose gum, hydroxypropyl cellulose, hydroxypropyl methylcellulose tragacanth, karaya, acacia, ghatti, beta-glucan, psyllium husk, inulin, chitosan, curdlan, dextran, potato starch, tapioca/arrowroot starch, hyaluronic acid, fibrin, carrageenan, guar gum, inulin, konjac, oat bran, pectin, locust bean gum, xanthan gum, soy protein, wheat protein, pea protein, chickpea protein, almond protein, rice protein, hemp protein, quinoa protein, sunflower protein, potato protein, algae protein, yeast protein, bacterial protein, corn protein, silk protein, cellulose derivatives, pullulan, hydrolysates of any of the aforementioned proteins, and combinations thereof.

64. The cultured adipose tissue of cases 62 or 63, wherein at least a portion of the adipose cells are cross-linked with alginate.

65. A cultured adipose tissue comprising adipose cells cross-linked together, wherein the cultured adipose tissue has a three-dimensional (3D) shape and a size on the macroscale.

66. The cultured adipose tissue of case 65, wherein the adipose cells are cross-linked using an enzyme selected from the group consisting of a transglutaminase, a tyrosinase, a peroxidase, a laccase, a sortase, a subtilisin, and lysyl oxidase.

67. The cultured adipose tissue of case 66, wherein the adipose cells are cross-linked with transglutaminase.

68. The cultured adipose tissue of case 65, wherein the adipose cells are cross-linked with a cross-linker selected from the group consisting of polymers functionalized with aldehyde groups, genipin, and phenolic compounds.

69. The cultured adipose tissue of case 68, wherein the polymers functionalized with aldehyde groups are selected from a group consisting of periodate oxidized pectin, dextran, chitosan, Arabic gum, sucrose, raffinose, stachyose, cyclodextrin, and starch.

70. The cultured adipose tissue of case 68, wherein the phenolic compounds are selected from the group consisting of caffeic acid, chlorogenic acid, caftaric acid, quercetin, and rutin.

71. The method or cultured adipose tissue of any one of the preceding cases, wherein the cultured adipose tissue is a food product.

72. The method or cultured adipose tissue of any one of the preceding cases, wherein the cultured adipose tissue is an ingredient of a food product.

73. The method or cultured adipose tissue of any one of the preceding cases, wherein one or more components of the adipose tissue are ingredients in a food product.

74. The method or cultured adipose tissue of any one of the preceding cases, wherein the cultured adipose tissue is produced without vascularization or perfusion.

75. A food product produced by the method of any one of cases 1-60.

76. The food product of case 75, wherein the cultured adipose tissue is integrated with other components to make a food product that includes the cultured adipose tissue.

77. The food product of case 75 or 76, further comprising shaping the cultured adipose tissue.

78. The food product of any one of cases 75-77, further comprising cooking, frying, and/or searing the cultured adipose tissue.

79. A composition produced by the method of any one of cases 1-60, wherein the composition is further processed to be used as a cosmetic, skin cream, lotion, soap, detergent, emulsifier, lubricant, fuel, and/or animal feed.

80. A system for executing the method of any one of cases 1-60 or 71-74, the system comprising feeding raw materials into a first vessel, wherein the first vessel provides a first output to optionally proceed to a second vessel and can provide waste via a waste output to be collected for reuse and recycling.

81. The system of case 80, comprising at least two vessels, wherein the at least two vessels are arranged to operate in series, in parallel, or in any combination of the two.

82. The system of case 80 or 81, further comprising extraction, filtration, and/or monitoring systems.

83. The system of any one of cases 80-82, wherein the raw material are from raw material sources that are sterile and/or temperature controlled.

84. The system of any one of cases 80-83, further comprising quality control facilities, cellular testing facilities, and/or appropriate facilities to confirm sterility.

85. The system of any one of cases 80-84, wherein the raw material sources are selected from the group comprising storage tanks, hoppers, reservoirs, and silos.

86. The system of any one of cases 80-85, wherein the adipose precursor cells are extracted from agricultural waste products.

87. The system of any one of cases 80-86, wherein at least one portion of the raw material sources is adapted to provide culture media.

88. The system of any one of cases 80-87, wherein the raw materials are transported throughout the system in a seed train, wherein cells move to progressively larger vessels.

89. The system of any one of cases 80-88, wherein the raw materials are isolated from the culture media by centrifugation, filtration, and/or micro-carrier.

90. The system of case 89, wherein centrifugation comprises counter flow centrifugation.

91. The system of case 89, wherein the micro-carrier is made out of pectin.

92. The system of any one of cases 80-91, further comprising at least one analytical device.

93. The system of case 92, wherein the at least one analytical device is selected from the group comprising an optical spectrophotometer, a gas chromatography system, and a mass spectrometry system.

1. A method for producing cultured adipose tissue, comprising:
growing adipogenic precursor cells in a first culture media;
differentiating the adipogenic precursor cells to adipose cells in a second culture media;
harvesting non-adhered adipose cells; and
aggregating the harvested non-adhered adipose cells to provide the cultured adipose tissue, optionally wherein the first culture media and the second culture media are the same media.

2. The method of claim 1, wherein growing the adipogenic precursor cells in the first culture media comprises seeding the adipogenic precursor cells into a bioreactor containing the first culture media and allowing the adipogenic precursor cells to proliferate in the bioreactor.

3-4. (canceled)

5. A method for producing cultured adipose tissue, comprising:
growing adipogenic precursor cells on a two-dimensional (2D) substrate;
differentiating the adipogenic precursor cells to adipose cells on the 2D substrate;
harvesting non-adhered adipose cells; and
aggregating the harvested non-adhered adipose cells to provide the cultured adipose tissue.

6. The method of claim 5, wherein the 2D substrate forms at least a portion of a conveyor belt.

7. The method of claim 5, wherein the method is performed continuously in an assembly line process.

8. (canceled)

9. The method of claim 1, wherein the adipogenic precursor cells are selected from the group consisting of mesenchymal stem cells, dedifferentiated fat cells, fibroblasts, fibroadipogenic progenitor cells and pluripotent stem cells.

10. The method of claim 1, wherein aggregating the harvested non-adhered adipose cells comprises mixing the harvested non-adhered adipose cells with a hydrogel or binder in a 3D mold.

11. (canceled)

12. The method of claim 1, wherein aggregating the harvested non-adhered adipose cells comprises mixing the harvested non-adhered adipose cells with alginate.

13. The method of claim 12, wherein mixing the harvested non-adhered adipose cells with alginate comprises:
adding calcium carbonate and glucono delta-lactone to a solution of the alginate; and
combining the harvested non-adhered adipose cells with the solution of the alginate in the 3D mold.

14. The method of claim 1, further comprising cross-linking the harvested non-adhered adipose cells.

15. The method of claim 14, wherein cross-linking the harvested non-adhered adipose cells comprises cross-linking the harvested non-adhered adipose cells using an enzyme selected from the group consisting of a transglutaminase, a tyrosinase, a peroxidase, a laccase, a sortase, a subtilisin, and lysyl oxidase.

16. The method of claim 14, wherein cross-linking the harvested non-adhered adipose cells comprises cross-linking the harvested non-adhered adipose cells using a cross-linker selected from the group consisting of polymers functionalized with aldehyde groups, genipin, and phenolic compounds, optionally wherein the polymers functionalized with aldehyde groups are selected from a group consisting of periodate oxidized pectin, dextran, chitosan, Arabic gum, sucrose, raffinose, stachyose, cyclodextrin, and starch and optionally wherein the phenolic compound is selected from the group consisting of caffeic acid, chlorogenic acid, caftaric acid, quercetin, and rutin.

17. The method of claim 1, wherein aggregating the harvested non-adhered adipose cells comprises adding a protein during the aggregation.

18. The method of claim 17, wherein the protein is selected from the group consisting of casein, gelatin, pea protein, soy protein, wheat protein, corn protein, chickpea protein, almond protein, rice protein, hemp protein, quinoa protein, sunflower protein, potato protein, algae protein, yeast protein, bacterial protein, mycoprotein, hydrolysates of any of the aforementioned proteins, and combinations thereof.

19. The method of claim 1, wherein the harvesting and/or the aggregating steps comprise flocculation or coagulation of the non-adhered adipose cells.

20-23. (canceled)

24. The method of claim 1, further comprising supplementing the adipose cells with methylated branched fatty acids.

25. (canceled)

26. The method of claim 1, further comprising monitoring the harvested non-adhered adipose cells for lipid accumulation prior to harvesting, wherein monitoring optionally comprises optical or electrical monitoring, wherein monitoring optionally comprises measuring lipid droplet size within a cell or within a population of cells.

27. (canceled)

28. The method of claim 1, further comprising determining the level of adipokine and/or adiponectin in the culture media prior to harvesting the non-adhered adipose cells.

29-32. (canceled)

33. The method of claim 1, further comprising reducing the nucleic acid content prior to harvesting the non-adhered adipose cells.

34. (canceled)

35. A cultured adipose tissue produced by the method of claim 1.

36-46. (canceled)
